(19) United States
(12) Patent Application Publication
(10) Pub. No.: US 2008/0227089 A1
(43) Pub. Date: Sep. 18, 2008

(54) Method of Diagnosing and/or Predicting the Development of an Allergic Disorder

(75) Inventors: Patrick Holt, Nedland WA (AU); Peter Sly, East Perth WA (AU); Anthony Bosco, Coogee WA (AU); Catherine Devitt, Como WA (AU); Katherine McKenna, Carine WA (AU)

Correspondence Address: SCHWEGMAN, LUNDBERG & WOESSNER, P.A., P.O. BOX 2938, MINNEAPOLIS, MN, 55402, US

(73) Assignee: Telethon Institute for Child Health Research

(21) Appl. No.: 11/662,026

(22) PCT Filed: Aug. 31, 2005
(86) PCT No.: PCT/AU05/01326

§ 371 Date: Nov. 13, 2007

Related U.S. Application Data
(60) Provisional application No. 60/607,699, filed on Sep. 7, 2004.

(30) Foreign Application Priority Data
Sep. 7, 2004 (AU) 2004905097

(51) Int. Cl. C12Q 1/68

(52) U.S. Cl. 435/6

(57) ABSTRACT

The present invention relates to methods for diagnosing an allergic disorder, predicting the development of an allergic disorder in an animal, monitoring the progress of therapy targeted at an allergic disorder, classification of the allergic disorder into one or more clinical/immunological phenotypes, and/or determining the potentional responsiveness of individual animals suffering from or at risk of an allergic disorder to particular forms of therapy. In particular, the present invention relates to a method of diagnosing and/or predicting the development of an allergic disorder in an animal, comprising the step of analysing a biological sample from the animal to determine the level of activation of one or more allergy-associated genes, in which the level of activation is diagnostic of the allergic disorder or predicative of the relative risk for the development of an allergic disorder in the animal.

### FIELD OF THE INVENTION

The present invention relates to methods for diagnosing an allergic disorder, predicting the development of an allergic disorder in an animal, monitoring the progress of therapy targeted at an allergic disorder, classification of the allergic disorder into one or more clinical/immunological phenotypes, and/or determining the potential responsiveness of individual animals suffering from or at risk of an allergic disorder to particular forms of therapy.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein; this reference does not constitute an admission that any of these documents forms part of the common general knowledge in the art, in Australia or in any other country.

Allergic disorders such as asthma, atopic dermatitis, hyper-IgE syndrome and allergic rhinitis represent some of the most common and best-characterised immune disorders in humans. Allergic disorders affect roughly 20 percent of all individuals in the United States.

However, while there are a number of clinical test procedures for assessing allergies (see generally American College of Physicians, “Allergy Testing,” Ann. Intern. Med. (1989) 110:317-320; Bousquet (1988) “In Vivo Methods for Study of Allergy: Skin Tests, Techniques, and Interpretation,” Allergy, Principles and Practice, 3rd Ed., Middleton et al., Eds., CV Mosby Co., St. Louis, Mo., pp. 419-436; and Van Arsdel et al. (1989) Ann. Intern. Med. 110:304-312), the methods available for early diagnosis of allergy, for predicting whether an individual will develop allergy, or for determining which subtype of allergy affects an individual patient are imprecise and subject to high levels of patient-to-patient variability.

The underlying reason for this variability is that allergic disorders are multifactorial in origin, and involve the operation within individual patients of different combinations of inflammatory mechanisms, driven by the products of a large number of different genes. However, the currently-available tests measure the products of only a very restricted range of genes. In other words, all of the currently-available immunological or clinical tests for allergy provide only superficial information about an individual's current immunological status, and there are no methods which can reliably determine what type or subtype of allergy is affecting an individual patient.

Accordingly, there is a need for more precise non-invasive methods for diagnosing and/or predicting the development of allergic disorders, and for determining what type or subtype of allergy is being expressed.

### SUMMARY OF THE INVENTION

In a first aspect the invention provides a method of diagnosing and/or predicting the development of an allergic disorder in an animal, comprising the step of analysing a biological sample from the animal to determine the level of activation of one or more allergy-associated genes, in which the level of activation is diagnostic of the allergic disorder or predictive of the relative risk for the development of an allergic disorder in the animal. In the majority of cases the genes of interest show upregulation of expression; in some cases the genes are down regulated.

In a second aspect the invention provides a method of monitoring the progress of therapy of an allergic disorder in an animal undergoing the therapy, comprising the steps of:
(a) analysing one or more biological samples from the animal to determine the level of activation of one or more allergy-associated genes, and
(b) determining whether the level of activation changes during therapy,
wherein a change in the level of activation during therapy is an indication of the progress of the therapy.

In a third aspect the invention provides a method of determining the potential responsiveness of an individual animal suffering from an allergic disorder to a therapy for the allergic disorder, comprising the step of analysing a biological sample from the animal to determine the level of activation of one or more allergy-associated genes, wherein the level of activation predicts the potential responsiveness of the animal to the therapy.

In a fourth aspect the invention provides a method of predicting the risk of progression to severe and/or persistent allergy in an animal suffering from an allergic disorder, comprising the step of obtaining a biological sample from the animal and determining the level of mRNA transcripts from one or more allergy-associated genes in the sample, wherein the presence of the mRNA is associated with increased risk of progression to severe and/or persistent allergy.

In a fifth aspect the invention provides a method of determining the immunological phenotype of an allergic condition in an animal, comprising the steps of obtaining a biological sample from said animal and determining the level of one or more mRNA transcripts from allergy-associated genes in said sample, wherein the presence of the mRNA is associated with or contributes to a specific allergy phenotype.

In a sixth aspect the invention provides a method of identifying an animal capable of responding to specific immunotherapy, comprising the steps of obtaining a biological sample from the animal, and determining the level of activation of an allergy-associated gene in the sample, in which the level of activation is predictive of the ability of the animal to respond to immunotherapy.

In an seventh aspect the invention provides a method of monitoring the response of an allergic animal to immunotherapy, comprising the steps of:
(a) analysing one or more biological samples from the animal to determine the level of activation of one or more specific genes encoded by said genes;
(b) subjecting the animal to immunotherapy;
(c) analysing one or more further biological samples from said animal to determine if the level of activation changes during immunotherapy;
wherein a change in the level of activation during immunotherapy is an indication of the responsiveness of the animal to the immunotherapy.

In an eighth aspect the invention provides a method of monitoring the effectiveness of a treatment for the reducing the severity of an allergic disorder, comprising the steps of:
(a) analysing one or more biological samples from an allergic animal to determine the level of activation of one or more allergy-associated genes;
(b) subjecting the animal to the treatment; and
(c) analysing one or more further biological samples from the animal to determine if the level of activation changes during treatment;

wherein a change in the level of activation during treatment is an indication of the responsiveness of the animal to the treatment.

In all aspects of the invention the gene may be any gene associated with an allergic disorder. Preferably the gene is one or more selected from the group consisting of cig5, IFIT4, LAMP3, DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 and CISH, or which comprises a sequence selected from the group consisting of sequences identified by probes 243610_at on human chromosome 9q21.13 at locus 138255, 1556097_at on human chromosome 15q25.2 and 242743_at on human chromosome 16p12.1 respectively, or is a combination of two or more of these genes.

In all aspects of the invention the step of determining the level of activation of the gene can be performed by any method known in the art. Preferably this is carried out by detecting the presence of mRNA by reverse transcription polymerase chain reaction (RT-PCR), or using specific nucleic acid arrays utilising microchip technology.

In some embodiments mRNA is detected using primers specific for a region of one or more genes selected from the group consisting of cig5, IFIT4, LAMP3, DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 and CISH, or which comprises a sequence selected from the group consisting of sequences identified by probes 243610_at on human chromosome 9q21.13 at locus 138255, 1556097_at on human chromosome 15q25.2 and 242743_at on human chromosome 16p12.1 respectively.

The primer can be selected from the group consisting of the following sets of primer pairs:

| | |
|---|---|
| cig5 forward: | |
| 5′CAAGACCGGGGAGAATACCTG3′ | (SEQ ID NO:1) |
| cig5 reverse: | |
| 5′GCGAGAATGTCCAAATACTCACC3′ | (SEQ ID NO:2) |
| IFIT4 forward: | |
| 5′GAGTGAGGTCACCAAGAATTC3′ | (SEQ ID NO:3) |
| IFIT4 reverse: | |
| 5′CACTCTATCTTCTAGATCCCTTGAGA3′ | (SEQ ID NO:4) |
| LAMP3 forward: | |
| 5′GCGTCCCTGGCCGTAATTT3′ | (SEQ ID NO:5) |
| LAMP3 reverse: | |
| 5′TGGTTGCTTAGCTGGTTGCT3′ | (SEQ ID NO:6) |
| DACT1 forward: | |
| 5′AACTCGGTGTTCAGTGAGTGT3′ | (SEQ ID NO:7) |
| DACT1 reverse: | |
| 5′GGAGAGGGAACGGCAAACT3′ | (SEQ ID NO:8) |
| IL17RB forward: | |
| 5′TGTGGAGGCACGAAAGGAT3′ | (SEQ ID NO:9) |
| IL17RB reverse: | |
| GATGGGTAAACCACAAGAACCT3′ | (SEQ ID NO:10) |
| KRT1 forward: | |
| 5′TCAATCTCGGTTGGATTCGGA3′ | (SEQ ID NO:11) |
| KRT1 reverse: | |
| 5′CTGCTTGGTAGAGTGCTGTAAGG3′ | (SEQ ID NO:12) |
| LNPEP forward: | |
| 5′TTCACCAATGATCGGCTTCAG3′ | (SEQ ID NO:13) |
| LNPEP reverse: | |
| 5′CTCCATCTCATGCTCACCAAG3′ | (SEQ ID NO:14) |
| MAL forward: | |
| 5′TCGTGGGTGCTGTGTTTACTCT3′ | (SEQ ID NO:15) |
| MAL reverse: | |
| 5′CAGTTGGAGGTTAGACACAGCAA3′ | (SEQ ID NO:16) |
| NCOA3 forward: | |
| 5′CCTGTCTCAGCCACGAGCTA3′ | (SEQ ID NO:17) |
| NCOA3 reverse: | |
| 5′TCCTGAAAGATCATGTCTGGTAA3′ | (SEQ ID NO:18) |
| OAZ forward: | |
| 5′TCAATTTACACCTGCGATCACTG3′ | (SEQ ID NO:19) |
| OAZ reverse: | |
| 5′GTTGTGGGTCGTCATCACCA3′ | (SEQ ID NO:20) |
| PECAM1 forward: | |
| 5′AGTCCAGATAGTCGTATGTGAAATGC3′ | (SEQ ID NO:21) |
| PECAM1 reverse: | |
| GGTCTGTCCTTTTATGACCTCAAAC3′ | (SEQ ID NO:22) |
| PLXDC1 forward: | |
| 5′CCTGGGCATGTGTCAGAGC3′ | (SEQ ID NO:23) |
| PLXDC1 reverse: | |
| 5′GGTGTTGGAGAGTATTGTGTGG3′ | (SEQ ID NO:24) |
| RASGRP3 forward: | |
| 5′TCAGCCTCATCGACATATCCA3′ | (SEQ ID NO:25) |
| RASGRP3 reverse: | |
| 5′TCAGCCAATTCAATGGGCTCC3′ | (SEQ ID NO:26) |
| SLC39A8 forward: | |
| 5′GCAGTCTTACAGCAATTGAACTTT3′ | (SEQ ID NO:27) |
| SLC39A8 reverse: | |
| 5′CCATATCCCCAAACTTCTGAA3′ | (SEQ ID NO:28) |
| XBP1 forward: | |
| 5′GTAGATTTAGAAGAAGAGAACCAAAAAC3′ | (SEQ ID NO:29) |
| XBP1 reverse: | |
| 5′CCCAAGCGCTGTCTTAACTC3′ | (SEQ ID NO:30) |
| NDFIP2 forward: | |
| 5′AGTGGGGAATGATGGCATTTT3′ | (SEQ ID NO:31) |
| NDFIP2 reverse: | |
| AAATCCGCAGATAGCACCA3′ | (SEQ ID NO:32) |
| RAB27B forward: | |
| 5′CAGAAACTGGATGAGCCAACT3′ | (SEQ ID NO:33) |
| RAB27B reverse: | |
| 5′GACTTCCCTCTGATCTGGTAGG3′ | (SEQ ID NO:34) |
| 243610_at forward: | |
| 5′TGCATTGACAACGTACTCAGAA3′ | (SEQ ID NO:35) |
| 243610_at reverse: | |
| 5′TCATCTTGACAGGGATAAGCAT3′ | (SEQ ID NO:36) |
| GNG8 forward: | |
| 5′GAACATCGACCGCATGAAGGT3′ | (SEQ ID NO:37) |
| GNG8 reverse: | |
| 5′AGAACACAAAAGAGGCGCTTG3′ | (SEQ ID NO:38) |
| GJB2 forward: | |
| 5′GCTTCCTCCCGACGCAGA3′ | (SEQ ID NO:39) |
| GJB2 reverse: | |
| 5′AACGAGGATCATAATGCGAAA3′ | (SEQ ID NO:40) |
| 1556097_at forward: | |
| 5′TCTTATTTCACTTTCTCAACTCATCA3′ | (SEQ ID NO:41) |
| 1556097_at reverse: | |
| 5′GGCATAACCTGAATGTATAATTCAA3′ | (SEQ ID NO:42) |
| 242743_at forward: | |
| 5′GAAAAAGCTGTTGAGTGAAGAAGACT3′ | (SEQ ID NO:43) |
| 242743_at reverse: | |
| 5′TGCAGGATGAGCAATGCTGAGA3′ | (SEQ ID NO:44) |
| and | |
| CISH forward: | |
| 5′GGGAATCTGGCTGGTATTGG3′ | (SEQ ID NO:45) |
| CISH reverse: | |
| 5′TTCTGGCATCTTCTGCAGGTGTT3′. | (SEQ ID NO:46) |

Alternatively the level of activation is determined by detection of the protein encoded by the mRNA, for example using ELISA, proteomic arrays, or intracellular staining as detected by flow cytometry. All of these methods are well known in the art.

It will be appreciated that in some cases the gene will be inactive, i.e. will not be transcribed or translated to a significant extent, while in other cases the expression of the gene will be modulated, i.e. it will be upregulated or down regulated.

For all aspects of the invention, in one embodiment the allergy-associated gene is upregulated in allergen-challenged PBMC from atopic individuals but is upregulated weakly if at all in PBMC from individuals who are not allergic to that allergen. Preferably in this embodiment the gene is one or more selected from the group consisting of DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 and CISH, and genes which comprise a sequence selected from the group consisting of sequences identified by probes 243610_at on human chromosome 9q21.13 at locus 138255, 1556097_at on human chromosome 15q25.2 and 242743_at on human chromosome 16p12.1 respectively. More preferably the gene is upregulated in atopic individuals and down-regulated in non-atopic individuals. Even more preferably the gene is KRT₁, PECAM₁, PLXDC₁, DACT1 or MAL.

In an alternative embodiment the allergy-associated gene is down-regulated in allergen-challenged PBMC from non-atopic individuals, but is not down-regulated in PBMC from atopic individuals. Preferably in this embodiment the gene is selected from the group consisting of cig5, IFIT4 and LAMP3.

The biological sample can be any biological material isolated from an atopic or non-atopic mammal, including blood, bone marrow, plasma, serum, lymph, cerebrospinal fluid, or a cellular or fluid component thereof; external sections of the skin, respiratory, intestinal, and genitourinary tracts; other secretions such as tears, saliva, or milk; tissue or organ biopsy samples; or cultured cells or cell culture supernatants. Preferably the biological sample is blood or lymph, or a cellular or fluid component thereof. More preferably the biological sample is bone marrow-derived mononuclear cells from peripheral blood (PBMC), which have been stimulated by in vitro exposure to one or more allergens to which the mammal is allergic. The skilled person will readily be able to determine whether prior exposure to allergen in vitro is advantageous in a particular case. This may depend on the nature of the biological sample.

The mammal may be a human, or may be a domestic, companion or zoo animal. While it is particularly contemplated that the compounds of the invention are suitable for use in medical treatment of humans, they are also applicable to veterinary treatment, including treatment of companion animals such as dogs and cats, and domestic animals such as horses, cattle and sheep, or zoo animals such as non-human primates, felids, canids, bovids, and ungulates.

Methods and pharmaceutical carriers for preparation of pharmaceutical or diagnostic compositions are well known in the art, as set out in textbooks such as Remington's Pharmaceutical Sciences, 20th Edition, Williams & Wilkins, Pennsylvania, USA.

### BRIEF DESCRIPTION OF THE FIGURES

FIG. 1 shows the results of the CD69, CFSE, CD4 and CD8 kinetic experiments performed on HGU133A arrays, and the PMBC kinetic experiment performed on HGU133plus2 arrays. t16, t24 and t48 represent 16, 24 and 48 hours of culture.

FIG. 2 shows a comparison of the level of expression of the gene cig5 in Cb4 cells from individuals allergic to HDM and from non-allergic individuals.

FIG. 3 shows a comparison of the level of expression of the gene IFIT4 in CD4 cells from individuals allergic to HDM and from non-allergic individuals.

FIG. 4 shows a comparison of the level of expression of the gene LAMP3 in CD4 cells from individuals allergic to HDM and from non-allergic individuals.

FIG. 5 shows a comparison of the level of expression of the gene DACT1 in CD4 cells from individuals allergic to HDM and from non-allergic individuals.

FIG. 6 shows a comparison of the level of expression of the gene IL17RB in CD4 cells from individuals allergic to HDM and from non-allergic individuals.

FIG. 7 shows a comparison of the level of expression of the gene KRT1 in CD4 cells from individuals allergic to HDM and from non-allergic individuals.

FIG. 8 shows a comparison of the level of expression of the gene LNPEP expression in CD4 cells from individuals allergic to HDM and from non-allergic individuals.

FIG. 9 shows a comparison of the level of expression of the gene MAL in CD4 cells from individuals allergic to HDM and from non-allergic individuals.

FIG. 10 shows a comparison of the level of expression of the gene NCOA3 in CD4 cells from individuals allergic to HDM and from non-allergic individuals.

FIG. 11 shows a comparison of the level of expression of the gene OAZ in CD4 cells from individuals allergic to HDM and from non-allergic individuals.

FIG. 12 shows a comparison of the level of expression of the gene PECAM1 in CD4 cells from individuals allergic to HDM and from non-allergic individuals.

FIG. 13 shows a comparison of the level of expression of the gene PLXDC1 in CD4 cells from individuals allergic to HDM and from non-allergic individuals.

FIG. 14 shows a comparison of the level of expression of the gene RASGRP3 in CD4 cells from individuals allergic to HDM and from non-allergic individuals.

FIG. 15 shows a comparison of the level of expression of the gene SLC39A8 in CD4 cells from individuals allergic to HDM and from non-allergic individuals.

FIG. 16 shows a comparison of the level of expression of the gene XBP1 in CD4 cells from individuals allergic to HDM and from non-allergic individuals.

FIG. 17 shows a comparison of the level of expression of the gene CISH in CD4 cells from individuals allergic to HDM and from non-allergic individuals.

FIG. 18 shows a comparison of the level of expression of the gene cig5 in PBMC from individuals allergic to HDM and from non-allergic individuals.

FIG. 19 shows a comparison of the level of expression of the gene IFIT4 in PBMC from individuals allergic to HDM and from non-allergic individuals.

FIG. 20 shows a comparison of the level of expression of the gene LAMP3 in PBMC from individuals allergic to HDM and from non-allergic individuals.

FIG. 21 shows a comparison of the level of expression of the gene DACT1 in PBMC from individuals allergic to HDM and from non-allergic individuals.

FIG. 22 shows a comparison of the level of expression of the gene IL17RB in PBMC from individuals allergic to HDM and from non-allergic individuals.

FIG. 23 shows a comparison of the level of expression of the gene KRT1 expression in PBMC from individuals allergic to HDM and from non-allergic individuals.

FIG. 24 shows a comparison of the level of expression of the gene LNPEP expression in PBMC from individuals allergic to HDM and from non-allergic individuals.

FIG. 25 shows a comparison of the level of expression of the gene MAL expression in PBMC from individuals allergic to HDM and from non-allergic individuals.

FIG. 26 shows a comparison of the level of expression of the gene NCOA3 expression in PBMC from individuals allergic to HDM and from non-allergic individuals.

FIG. 27 shows a comparison of the level of expression of the gene OAZ expression in PBMC from individuals allergic to HDM and from non-allergic individuals.

FIG. 28 shows a comparison of the level of expression of the gene PECAM1 expression in PBMC from individuals allergic to HDM and from non-allergic individuals.

FIG. 29 shows a comparison of the level of expression of the gene PLXDC1 expression in PBMC from individuals allergic to HDM and from non-allergic individuals.

FIG. 30 shows a comparison of the level of expression of the gene RASGRP3 expression in PBMC from individuals allergic to HDM and from non-allergic individuals.

FIG. 31 shows a comparison of the level of expression of the gene SLC39A8 expression in PBMC from individuals allergic to HDM and from non-allergic individuals.

FIG. 32 shows a comparison of the level of expression of the gene XBP1 expression in PBMC from individuals allergic to HDM and from non-allergic individuals.

FIG. 33 shows a comparison of the level of expression of the gene NDFIP2 expression in PBMC from individuals allergic to HDM and from non-allergic individuals.

FIG. 34 shows a comparison of the level of expression of the gene RAB27B expression in PBMC from individuals allergic to HDM and from non-allergic individuals.

FIG. 35 shows a comparison of the level of expression of the gene 242743_AT expression in PBMC from individuals allergic to HDM and from non-allergic individuals.

FIG. 36 shows a comparison of the level of expression of the gene GNG8 expression in PBMC from individuals allergic to HDM and from non-allergic individuals.

FIG. 37 shows a comparison of the level of expression of the gene GJB2 expression in PBMC from individuals allergic to HDM and from non-allergic individuals.

FIG. 38 shows a comparison of the level of expression of the gene 1556097 expression in PBMC from individuals allergic to HDM and from non-allergic individuals.

FIG. 39 shows a comparison of the level of expression of the gene 243610_AT expression in PBMC from individuals allergic to HDM and from non-allergic individuals.

FIG. 40 shows a comparison of the level of expression of the gene CISH expression in PBMC from individuals allergic to HDM and from non-allergic individuals.

FIG. 41 shows a comparison of the level of expression of IL4 mRNA expression at 16 hours post-stimulation for CD4+ cells from individuals allergic to HDM and from non-allergic individuals as assessed by quantitative real-time PCR.

FIG. 42 shows a comparison of the level of expression of DACT1 mRNA expression at 16 hours post-stimulation for CD4+ cells from individuals allergic to HDM and from non-allergic individuals as assessed by quantitative real-time PCR.

FIG. 43 shows a comparison of the level of expression of LAMP3 mRNA expression at 16 hours post-stimulation for CD4+ cells from individuals allergic to HDM and from non-allergic individuals as assessed by quantitative real-time PCR.

FIG. 44 shows a comparison of the level of expression of PLXDC1 mRNA expression at 16 hours post-stimulation for CD4+ cells from individuals allergic to HDM and from non-allergic individuals as assessed by quantitative real-time PCR.

FIG. 45 shows a comparison of the level of expression of PLXDC1 mRNA expression at 48 hours post-stimulation for CD4+ cells from individuals allergic to HDM and from non-allergic individuals as assessed by quantitative real-time PCR.

FIG. 46 shows a comparison of the level of expression of cig5 mRNA expression at 16 hours post-stimulation for CD4+ cells from individuals allergic to HDM and from non-allergic individuals as assessed by quantitative real-time PCR.

FIG. 47 shows a comparison of the level of expression of IFIT4 mRNA expression at 16 hours post-stimulation for CD4+ cells from individuals allergic to HDM and from non-allergic individuals as assessed by quantitative real-time PCR.

FIG. 48 shows a comparison of the level of expression of MAL mRNA expression at 16 hours post-stimulation for CD4+ cells from individuals allergic to HDM and from non-allergic individuals as assessed by quantitative real-time PCR.

FIG. 49 shows a comparison of the level of expression of PECAM1 mRNA expression at 16 hours post-stimulation for CD4+ cells from individuals allergic to HDM and from non-allergic individuals -as assessed by quantitative real-time PCR.

FIG. 50 shows a comparison of the level of expression of SLC39A8 mRNA expression at 16 hours post-stimulation for CD4+ cells from individuals allergic to HDM and from non-allergic individuals as assessed by quantitative real-time PCR.

FIG. 51 shows a comparison of the level of expression of XBP1 mRNA expression at 16 hours post-stimulation for CD4+ cells from individuals allergic to HDM and from non-allergic individuals as assessed by quantitative real-time PCR.

FIG. 52 shows a comparison of the level of expression of NDFIP2 mRNA expression at 16 hours post-stimulation for CD4+ cells from individuals allergic to HDM and from non-allergic individuals as assessed by quantitative real-time PCR.

FIG. 53 shows a comparison of the level of expression of 243610_at CISH mRNA expression at 16 hours post-stimulation for CD4+ cells from individuals allergic to HDM and from non-allergic individuals as assessed by quantitative real-time PCR.

FIG. 54 shows a comparison of the level of expression of CISH mRNA expression at 16 hours post-stimulation for CD4+ cells from individuals allergic to HDM and from non-allergic individuals as assessed by quantitative real-time PCR.

FIG. 55 shows a comparison of the level of expression of NCOA3 mRNA expression at 48 hours post-stimulation for CD4+ cells from individuals allergic to HDM and from non-allergic individuals as assessed by quantitative real-time PCR.

FIG. 56 shows a comparison of the level of expression of NDFIP2 mRNA expression at 16 hours post-stimulation for PBMC cells from individuals allergic to HDM and from non-allergic individuals as assessed by quantitative real-time PCR.

FIG. 57 shows a comparison of the level of expression of RAB27B mRNA expression at 16 hours post-stimulation for PBMC cells from individuals allergic to HDM and from non-allergic individuals as assessed by quantitative real-time PCR.

FIG. 58 shows a comparison of the level of expression of 243610_at mRNA expression at 16 hours post-stimulation for PBMC cells from individuals allergic to HDM and from non-allergic individuals as assessed by quantitative real-time PCR.

FIG. 59 shows a comparison of the level of expression of GNG8 mRNA expression at 16 hours post-stimulation for PBMC cells from individuals allergic to HDM and from non-allergic individuals as assessed by quantitative real-time PCR.

FIG. 60 shows a comparison of the level of expression of GJB2 mRNA expression at 16 hours post-stimulation for PBMC cells from individuals allergic to HDM and from non-allergic individuals as assessed by quantitative real-time PCR.

FIG. 61 shows a comparison of the level of expression of 1556097_at mRNA expression at 16 hours post-stimulation for PBMC cells from individuals allergic to HDM and from non-allergic individuals as assessed by quantitative real-time PCR.

FIG. 62 shows a comparison of the level of expression of 242743_at mRNA expression at 16 hours post-stimulation for CD4⁺ cells from individuals allergic to HDM and from non-allergic individuals as assessed by quantitative real-time PCR.

FIG. 63 shows a comparison of the level of expression of 242743_at mRNA expression at 16 hours post-stimulation for PBMC cells from individuals allergic to HDM and from non-allergic individuals as assessed by quantitative real-time PCR.

### DETAILED DESCRIPTION OF THE INVENTION

Before describing the present invention in detail, it is to be understood that this invention is not limited to particularly exemplified methods of diagnosis and may, of course, vary. It is also to be understood that the terminology used herein is for the purpose of describing particular embodiments of the invention only, and is not intended to be limiting.

All publications, patents and patent applications cited herein are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

Furthermore, the practice of the invention employs, unless otherwise indicated, conventional immunological techniques, chemistry and pharmacology within the skill of the art. Such techniques are well known to the skilled worker, and are explained fully in the literature. See, e.g., Colignan, Dunn, Ploegh, Speicher and Wingfield “Current protocols in Protein Science” (1999) Volume I and II (John Wiley & Sons Inc.); and Bailey, J. E. and Ollis, D. F., Biochemical Engineering Fundamentals, McGraw-Hill Book Company, NY, 1986.

Abbreviations used herein are as follows:

ELISA enzyme-linked immunoadsorbent assay

HDM house dust mite

IL-4 interleukin 4

PCR polymerase chain reaction

RT-PCR reverse transcriptase polymerase chain reaction

In the claims of this application and in the description of the invention, except where the context requires otherwise due to express language or necessary implication, the words “comprise” or variations such as “comprises” or “comprising” are used in an inclusive sense, i.e. to specify the presence of the stated features but not to preclude the presence or addition of further features in various embodiments of the invention.

It must be noted that as used herein and in the appended claims, the singular forms “a,” “an,” and “the” include plural reference unless the context clearly dictates otherwise. Thus, for example, a reference to “a gene” includes a plurality of such genes, and a reference to “an allergy” is a reference to one or more allergies, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

Where a range of values is expressed, it will be clearly understood that this range encompasses the upper and lower limits of the range, and all values in between these limits.

In its broadest sense, the present invention relates to a method for diagnosing and/or predicting the development of an allergic disorder. As used herein, the terms “diagnosis” or “diagnosing” refers to the method of distinguishing one allergic disorder from another allergic disorder, or determining whether an allergic disorder is present in an animal (atopic) relative to the “normal” or “non-allergic” (non-atopic) state, and/or determining the nature of an allergic disorder.

As used herein, the term “atopic” or “allergic” refers to an animal which has an allergic reaction. Conversely a “non-atopic” animal is one which does not have an allergic reaction. Allergy is conventionally diagnosed by skin tests such as the skin prick, intradermal or skin patch test, by determination of serum IgE antibody by radioallergosorbent testing (RAST), or by ELISA or related methods.

Allergies are caused by allergens, which may be present in a wide variety of sources, including but not limited to pollens or other plant components, dust, moulds or fungi, foods, animal or bird danders, insect venoms, or chemicals.

As used herein, the terms “allergic disorder” or “allergic condition” refer to an abnormal biological function characterised by either an increased responsiveness of the trachea and bronchi to various stimuli or by a disorder involving inflammation at these or other sites in response to allergen exposure. The symptoms associated with these allergic disorders include, but are not limited to, cold, cold-like, and/or “flu-like” symptoms, cough, dermal irritation, dyspnea, lacrimation, rhinorrhea, sneezing and wheezing, and skin manifestations. Allergic disorders are also often associated with an increase in Th2 cytokines such as IL-4, IL-4R, IL-5, IL-9 and IL-13. Examples of allergic disorders include, but are not limited to, asthma, atopic dermatitis, bronchoconstriction, chronic airway inflammation, allergic contact dermatitis, eczema, food allergy, hay fever, hyper-IgE syndrome, rhinitis, and allergic urticaria.

As stated above, the invention also relates to a method for predicting the development of an allergic disorder. The term “predicting the development” when used with reference to an allergic disorder means that an animal does not currently have an allergic disorder or does not have clinical symptoms of an allergic disorder, but has a propensity to develop an allergic disorder. The terms “propensity to develop an allergic disorder,” “predisposition” or “susceptibility” or any similar phrases, mean that an animal which can develop allergy has certain “allergy-associated genes” which are “activated” such that they are predictive of an animal's risk of developing a particular disorder (e.g. asthma). The activation of these “allergy-associated genes” in an animal predisposed to an allergic disorder in comparison to healthy individual animals is predictive of the development of an allergic disorder even in pre-symptomatic animals.

In one embodiment, the term “predicting the development” also includes animals which have an allergic disorder, and the methods disclosed herein are used to assess the severity of the disorder or to predict its progression more accurately.

Without wishing to be bound by any particular theory or hypothesis, the inventors have demonstrated that a number of genes, including some which had not previously been considered to be associated with allergic disorders, are activated in allergen-stimulated peripheral blood mononuclear cells (PMBC) from animals which have an allergic disorder. However, these genes are activated to a lesser extent in animals which do not have an allergic disorder. For example, the inventors have noted that genes such as DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 and CISH or combinations of two or more of these genes are strongly activated in house dust mite (HDM)-stimulated PMBC from humans allergic to house dust mite, whereas these genes are activated only weakly or not at all in PMBC from humans who are not allergic to house dust mite. In contrast, other genes such as cig5, IFIT4 and LAMP3 are actively down-regulated in HDM-stimulated PBMC from non-atopic individuals (normal individuals), whereas these genes are down-regulated only weakly or not at all in corresponding PBMC samples from atopic (“allergic”) individuals. These genes are still considered to be indicative of the non-atopic phenotype, and they are also considered to be representative of “protective” genes i.e. the products of these genes in some way provides protection against the development of allergy.

Accordingly, in one embodiment, the terms “allergy-associated genes” or “allergy-specific genes”, which are used herein interchangeably, refer to genes which are either typically associated with an allergic disorder or are shown to be associated with an allergic disorder in that an animal exhibiting clinical symptoms of an allergic disorder possesses a gene which is activated in the presence of a stimulating compound or allergen, wherein the level of activation is different to that of a non-allergic animal.

The term “activated” as used herein means that the gene is actively being transcribed in an animal, i.e. the corresponding mRNA or the protein encoded by that mRNA can be detected.

The term “mammal” as used herein includes, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs. The terms do not denote a particular age, and thus both adult and immature individuals are intended to be covered. The methods described herein are intended for use in any of the above mammalian species, since the immune systems of all of these mammals operate similarly.

Thus the invention encompasses the diagnosis of an allergic disorder or the prediction of the development of an allergic disorder in any mammal including a human, as well as those mammals of economic and/or social importance to humans, including carnivores such as cats, dogs and larger felids and canids, swine such as pigs, hogs, and wild boars, ruminants such as cattle, oxen, sheep, giraffes, deer, goats, bison, and camels, and horses, and non-human primates such as apes and monkeys. Thus the invention encompasses the diagnosis of an allergic disorder of livestock, including, but not limited to, domesticated swine, ruminants, horses, and the like, and zoo or endangered animals.

The step of analysing whether or not an allergy-associated gene is activated can be carried out using any standard techniques known in the art. For example, techniques such as reverse transcription polymerase chain reaction (RT-PCR) or DNA array analysis, ELISA, proteomic arrays, or intracellular staining as detected by flow cytometry may be used.

The biological sample may be tested using the techniques described herein directly after isolation, or alternatively may be further processed in order to increase the quality of the data produced. In this regard, the inventors have noted from the literature that the selective expansion of allergen specific cells by initial stimulation with allergen to induce proliferation generates a “cell line” in which the frequency of the relevant cells is a log scale greater than that of the same cells in a biological sample directly isolated from an animal. If required, the cells can be further concentrated and purified by cloning the specific cells.

Accordingly, in one embodiment, a biological sample such as peripheral blood is taken from an animal which is suspected of, or susceptible to the development of an allergic disorder. The biological sample is then treated so as to substantially isolate leukocytes from the blood i.e. separate the leukocytes from (or otherwise substantially free from), other contaminant cells. The biological sample is then exposed to an environmental allergen. The term “environmental allergen” as used herein refers to allergens that are specifically associated with the development of allergic disorders. For example, allergens might include those of animals, including the house dust mite (e.g. Dermatophagoides pteronyssinus, Dermatophagoides farinae, Blomia tropicalis), such as the allergens der p1 (Scobie et al. (1994) Biochem. Soc. Trans. 22: 448S; Yssel et al. (1992) J. Immunol. 148: 738-745), der p2 (Chua et al. (1996) Clin. Exp. Allergy 26: 829-837), der p3 (Smith & Thomas (1996) Clin. Exp. Allergy 26: 571-579), der p5, der p V (Lin et al. (1994) J. Allergy Clin. Immunol. 94: 989-996), der p6 (Bennett & Thomas (1996) Clin. Exp. Allergy 26: 1150-1154), der p 7 (Shen et al. (1995) Clin. Exp. Allergy 25: 416-422), der f2 (Yuuki et al. (1997) Int. Arch. Allergy Immunol. 112: 44-48), der f3 (Nishiyama et al. (1995) FEBS Lett. 377: 62-66), der f7 (Shen et al. (1995) Clin. Exp. Allergy 25: 1000-1006); Mag 3 (Fujikawa et al. (1996) Mol. Immunol. 33: 311-319). Also of interest as allergens are the house dust mite allergens Tyr p2 (Eriksson et al. (1998) Eur. J. Biochem. 251: 443-447), Lep d1 (Schmidt et al. (1995) FEBS Lett. 370: 11-14), and glutathione S-transferase (O'Neill et al. (1995) Immunol Lett. 48: 103-107); the 25,589 KDa, 219 amino acid polypeptide with homology with glutathione S-transferases (O'Neill et al. (1994) Biochim. Biophys. Acta. 1219: 521-528); Blo t 5 (Arruda et al. (1995) Int. Arch. Allergy Immunol. 107: 456-457); bee venom phospholipase A2 (Carballido et al. (1994) J. Allergy Clin. Immunol. 93: 758-767; Jutel et al. (1995) J. Immunol. 154: 4187-4194); bovine dermal/dander antigens BDA 11 (Rautiainen et al. (1995) J. Invest. Dermatol. 105: 660-663) and BDA20 (Mantyjarvi et al. (1996) J. Allergy Clin. Immunol. 97: 1297-1303); the major horse allergen Equ c1 (Gregoire et al. (1996) J. Biol. Chem. 271: 32951-32959); Jumper ant M. pilosula allergen Myr p I and its homologous allergenic polypeptides Myr p2 (Donovan et al. (1996) Biochem. Mol. Biol. Int. 39: 877-885); 1-13, 14, 16 kDa allergens of the mite Blomia tropicalis (Caraballo et al. (1996) J. Allergy Clin. Immunol. 98: 573-579); the cockroach allergens Bla g Bd90K.(Helm et al. (1996) J. Allergy Clin. Immunol. 98: 172-80) and Bla g 2 (Arruda et al. (1995) J. Biol. Chem. 270: 19563-19568); the cockroach Cr-PI allergens (Wu et al. (1996) J. Biol. Chem. 271: 17937-17943); fire ant venom allergen, Sol i 2 (Schmidt et al. (1996) J. Allergy Clin. Immunol. 98: 82-88); the insect Chironomus thummi major allergen Chi t 1-9 (Kipp et al. (1996) Int. Arch. Allergy Immunol. 110: 348-353); dog allergen Can f 1 or cat allergen Fel d 1 (Ingram et al. (1995) J. Allergy Clin. Immunol. 96: 449-456); albumin, derived, for example, from horse, dog or cat (Goubran Botros et al. (1996) Immunology 88: 340-347); deer allergens with the molecular mass of 22 kDa, 25 kDa or 60 kDa (Spitzauer et al. (1997) Clin. Exp. Allergy 27: 196-200); and the 20 kDa major allergen of cow (Ylonen et al. (1994) J. Allergy Clin. Immunol. 93: 851-858).

Pollen and grass allergens include, for example, Hor v9 (Astwood & Hill (1996) Gene 182: 53-62, Lig v1 (Batanero et al. (1996) Clin. Exp. Allergy 26: 1401-1410); Lol p 1 (Muller et al. (1996) Int. Arch. Allergy Immunol. 109: 352-355), Lol p II (Tamborini et al. (1995) Mol. Immunol. 32: 505-513), Lol pVA, Lol pVB (Ong et al. (1995) Mol. Immunol. 32: 295-302), Lol p 9 (Blaher et al. (1996) J. Allergy Clin. Immunol. 98: 124-132); Par J I (Costa et al. (1994) FEBS Lett. 341: 182-186; Sallusto et al. (1996) J. Allergy Clin. Immunol. 97: 627-637), Par j 2.0101 (Duro et al. (1996) FEBS Lett. 399: 295-298); Bet v1 (Faber et al. (1996) J. Biol. Chem. 271: 19243-19250), Bet v2 (Rihs et al. (1994) Int. Arch. Allergy Immunol. 105: 190-194); Dac g3 (Guerin-Marchand et al. (1996) Mol. Immunol. 33: 797-806); Phl p 1 (Petersen et al. (1995) J. Allergy Clin. Immunol. 95: 987-994), Phl p 5 (Muller et al. (1996) Int. Arch. Allergy Immunol. 109: 352-355), Phl p 6 (Petersen et al. (1995) Int. Arch. Allergy Immunol. 108: 55-59); Cry j I (Sone et al. (1994) Biochem. Biophys. Res. Commun. 199: 619-625), Cry j II (Namba et al. (1994) FEBS Lett. 353: 124-128); Cor a 1 (Schenk et al. (1994) Eur. J. Biochem. 224: 717-722); cyn d1 (Smith et al. (1996) J. Allergy Clin. Immunol. 98: 331-343), cyn d7 (Suphioglu et al. (1997) FEBS Lett. 402: 167-172); Pha a 1 and isoforms of Pha a 5 (Suphioglu and Singh (1995) Clin. Exp. Allergy 25: 853-865); Cha o 1 (Suzuki et al. (1996) Mol. Immunol. 33: 451-460); profilin derived, e.g, from timothy grass or birch pollen (Valenta et al. (1994) Biochem. Biophys. Res. Commun. 199: 106-118); P0149 (Wu et al. (1996) Plant Mol. Biol. 32: 1037-1042); Ory sl (Xu et al. (1995) Gene 164: 255-259); and Amb a V and Amb t 5 (Kim et al. (1996) Mol. Immunol. 33: 873-880; Zhu et al. (1995) J. Immunol. 155: 5064-5073).

Fungal allergens include, but are not limited to, the allergen, Cla h III, of Cladosporium herbarum (Zhang et al. (1995) J. Immunol. 154: 710-717); the allergen Psi c 2, a fungal cyclophilin, from the basidiomycete Psilocybe cubensis (Homer et al. (1995) Int. Arch. Allergy Immunol. 107: 298-300); hsp 70 cloned from a cDNA library of Cladosporium herbarum (Zhang et al. (1996) Clin Exp Allergy 26: 88-95); the 68 kDa allergen of Penicillium notatum (Shen et al. (1995) Clin. Exp. Allergy 26: 350-356); aldehyde dehydrogenase (ALDH) (Achatz et al. (1995) Mol Immunol. 32: 213-227); enolase (Achatz et al. (1995) Mol. Immunol. 32: 213-227); YCP4 (Id.); acidic ribosomal protein P2 (Id.).

Suitable food allergens include, for example, profilin (Rihs et al. (1994) Int. Arch. Allergy immunol. 105: 190-194); rice allergenic cDNAs belonging to the alpha-amylase/trypsin inhibitor gene family (Alvarez et al. (1995) Biochim Biophys Acta 1251: 201-204); the main olive allergen, Ole e I (Lombardero et al. (1994) Clin Exp Allergy 24: 765-770); Sin a 1, the major allergen from mustard (Gonzalez De La Pena et al. (1996) Eur J Biochem. 237: 827-832); parvalbumin, the major allergen of salmon (Lindstrom et al. (1996) Scand. J Immunol. 44: 335-344); apple allergens, such as the major allergen Mal d 1 (Vanek-Krebitz et al. (1995) Biochem. Biophys. Res. Commun. 214: 538-551); and peanut allergens, such as Ara h I (Burks et al. (1995) J Clin. Invest. 96: 1715-1721).

This step constitutes the stimulation phase of the described method. Following exposure to the environmental allergen the activation levels for the allergen associated genes are determined or measured.

Many techniques for detecting gene expression may be employed. Technology systems for pharmacogenomic assays have recently been reviewed (Koch, 2004, Nature Reviews Drug Discovery 3 749-761). Gene expression may be measured in a biological sample directly, for example, by conventional Southern blotting to quantitate DNA, or by Northern blotting to quantitate mRNA, using an appropriately labelled oligonucleotide hybridisation probe, based on the known sequences of the allergy-associated genes. Identification of mRNA from the allergy-associated genes within a mixture of various mRNAs is conveniently accomplished by the use of reverse transcriptase-polymerase chain reaction and an oligonucleotide hybridization probe that is labelled with a detectable moiety. Various labels may be employed, most commonly radioisotopes, particularly ³²P. However, other techniques may also be employed, such as using biotin-modified nucleotides for introduction into a polynucleotide. The biotin then serves as the site for binding to avidin or antibodies, which may be labelled with a wide variety of labels, such as radioisotopes, fluorophores, chromophores, or the like. Keller et al., DNA Probes, pp. 149-213 (Stockton Press, 1989). Alternatively, antibodies may be employed that can recognise specific duplexes, including DNA duplexes, RNA duplexes, and DNA-RNA hybrid duplexes or DNA-protein duplexes. The antibodies in turn may be labelled and the assay may be carried out where the duplex is bound to a surface, so that upon the formation of duplex on the surface, the presence of antibody bound to the duplex can be detected.

In one preferred method, mRNA in a biological sample is reverse transcribed to generate a cDNA strand. The cDNA may be amplified by conventional techniques, such as polymerase chain reaction, to provide sufficient amounts for analysis.

Amplification may also be used to determine whether a specific sequence is present, by using a primer that will specifically bind to the desired sequence, where the presence of an amplification product is indicative that a specific binding complex was formed. Alternatively, the mRNA sample is fractionated by electrophoresis, e.g. capillary or gel electrophoresis, transferred to a suitable support, e.g. nitrocellulose and then probed with a fragment of the transcription factor sequence. Other techniques may also find use, including oligonucleotide ligation assays, binding to solid-state arrays, etc. Detection of mRNA having the subject sequence is indicative gene expression of the transcription factor in the sample.

“Polymerase chain reaction,” or “PCR,” as used herein generally refers to a method for amplification of a desired nucleotide sequence in vitro, as described in U.S. Pat. No. 4,683,195. In general, the PCR method involves repeated cycles of primer extension synthesis, using two oligonucleotide primers capable of hybridizing preferentially to a template nucleic acid. Typically, the primers used in the PCR method will be complementary to nucleotide sequences within the template at both ends of or flanking the nucleotide sequence to be amplified, although primers complementary to the nucleotide sequence to be amplified also may be used. See Wang et al., 1990, In: PCR Protocols, pp. 70-75 (Academic Press,); Ochman, et al., 1990, In: PCR Protocols, pp. 219-227; Triglia, et al., 1988, Nucl. Acids Res. 16:8186.

“Oligonucleotides” are short-length, single- or double-stranded polydeoxynucleotides that are chemically synthesised by known methods (involving, for example, triester, phosphoramidite, or phosphonate chemistry), such as described by Engels et al., 1989, Agnew. Chem. mnt. Ed. Engl. 28:716-734. They are then purified, for example, by polyacrylamide gel electrophoresis.

As used herein, the term “PCR reagents” refers to the chemicals, apart from the target nucleic acid sequence, needed to perform the PCR process. These chemicals generally consist of five classes of components: (i) an aqueous buffer, (ii) a water soluble magnesium salt, (iii) at least four deoxyribonucleotide triphosphates (dNTPs), (iv) oligonucleotide primers (normally two primers for each target sequence, the sequences defining the 5′ ends of the two complementary strands of the double-stranded target sequence), and (v) a polynucleotide polymerase, preferably a DNA polymerase, more preferably a thermostable DNA polymerase, i.e. a DNA polymerase which can tolerate temperatures between 90° C. and 100° C. for a total time of at least 10 minutes without losing more than about half its activity.

The four conventional dNTPs are thymidine triphosphate (dTTP), deoxyadenosine triphosphate (dATP), deoxycytidine triphosphate (dCTP), and deoxyguanosine triphosphate (dGTP). These conventional triphosphates may be supplemented or replaced by dNTPs containing base analogues which Watson-Crick base pair like the conventional four bases, e.g. deoxyuridine triphosphate (dUTP).

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorexcein (6-FAM), 2′,7′-dimethoxy-4′,5′-dichloro-6-carboxyfluorescein (JOE), 6-carboxy-X-rhodamine(ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; as well as others. The label may be a two stage system, where the amplified DNA is conjugated to biotin, haptens, or the like having a high affinity binding partner, e.g. avidin, specific antibodies, etc., where the binding partner is conjugated to a detectable label. The label may be conjugated to one or both of the primers. Alternatively, the pool of nucleotides used in the amplification is labelled, so as to incorporate the label into the amplification product.

Accordingly, in one preferred embodiment, once the allergy-associated gene mRNA has been reverse transcribed and amplified by PCR, it is detected by various means including oligonucleotide probes. Oligonucleotide probes of the invention are DNA molecules that are sufficiently complementary to regions of contiguous nucleic acid residues within the allergy-associated gene nucleic acid to hybridise thereto, preferably under high stringency conditions. Exemplary probes include oligomers that are at least about 15 nucleic acid residues long and that are selected from any 15 or more contiguous residues of DNA of the present invention. Preferably, oligomeric probes used in the practice of the present invention are at least about 20 nucleic acid residues long. The present invention also contemplates oligomeric probes that are 150 nucleic acid residues long or longer. Those of ordinary skill in the art realise that nucleic hybridisation conditions for achieving the hybridisation of a probe of a particular length to polynucleotides of the present invention can readily be determined. Such manipulations to achieve optimal hybridisation conditions for probes of varying lengths are well known in the art. See, e.g., Sambrook et al., Molecular Cloning: A Laboratory Manual, Second Edition, Cold Spring Harbor (1989), incorporated herein by reference.

Preferably, oligomeric probes of the present invention are labelled to render them readily detectable. Detectable labels may be any species or moiety that may be detected either visually or with the aid of an instrument. Commonly used detectable labels are radioactive labels such as, for example, ³²P, ¹⁴C, ¹²⁵I, ³H, and ³⁵S. Examples of fluorescer-quencher pairs may be selected from xanthene dyes, including fluoresceins, and rhodamine dyes. Many suitable forms of these compounds are widely available commercially with substituents on their phenyl moieties which can be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl-6-naphthalene sulfonate. Other dyes include 3-phenyl-7-isocyanatocoumarin, acridines, such as 9-isothiocyanatoacridine acridine orange; N-(p-(2-benzoaxazolyl)phenyl)maleimide; benzoxadiazoles, stilbenes, pyrenes, and the like. Most preferably, the fluorescent compounds are selected from the group consisting of VIC, carboxy fluorescein (FAM), Lightcycler® 640 and Cy5.

Biotin-labelled nucleotides can be incorporated into DNA or RNA by such techniques as nick translation, chemical and enzymatic means, and the like. The biotinylated probes are detected after hybridisation, using indicating means such as avidin/streptavidin, fluorescent labelling agents, enzymes, colloidal gold conjugates, and the like. Nucleic acids may also be labelled with other fluorescent compounds, with immunodetectable fluorescent derivatives, with biotin analogues, and the like. Nucleic acids may also be labelled by means of attachment to a protein. Nucleic acids cross-linked to radioactive or fluorescent histone single-stranded binding protein may also be used. Those of ordinary skill in the art will recognise that there are other suitable methods for detecting oligomeric probes and other suitable detectable labels that are available for use in the practice of the present invention. Moreover, fluorescent residues can be incorporated into oligonucleotides during chemical synthesis.

Two DNA sequences are “substantially similar” when at least about 85%, preferably at least about 90%, and most preferably at least about 95%, of the nucleotides match over the defined length of the DNA sequences. Sequences that are substantially similar can be identified for example in a Southern hybridisation experiment performed under stringent conditions as defined for that particular system. Defining appropriate hybridisation conditions is within the skill of the art. See e.g., Maniatis et al., DNA Cloning, vols. I and II. Nucleic Acid Hybridisation. Briefly, “stringent conditions” for hybridisation or annealing of nucleic acid molecules are those that

(1) employ low ionic strength and high temperature for washing, for example, 0.015M NaCl/0.0015M sodium citrate/0.1% sodium dodecyl sulfate (SDS) at 50° C., or

(2) employ during hybridisation a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Another example is use of 50% formamide, 5×SSC (0.75M NaCl, 0.075M sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42° C., with washes at 42° C. in 0.2×SSC and 0.1% SDS.

In a particularly preferred embodiment the invention utilises a combined PCR and hybridisation probing system so as to take advantage of closed-tube or homogenous assay systems such as the use of FRET probes as disclosed in US patents (U.S. Pat. Nos. 6,140,054; 6,174,670), the entirety of which are also incorporated herein by reference. In one of its simplest configurations, the FRET or “fluorescent resonance energy transfer” approach employs two oligonucleotides which bind to adjacent sites on the same strand of the nucleic acid being amplified. One oligonucleotide is labelled with a donor fluorophore which absorbs light at a first wavelength and emits light in response, and the second is labelled with an acceptor fluorophore which is capable of fluorescence in response to the emitted light of the first donor (but not substantially by the light source exciting the first donor, and whose emission can be distinguished from that of the first fluorophore). In this configuration, the second or acceptor fluorophore shows a substantial increase in fluorescence when it is in close proximity to the first or donor fluorophore, such as occurs when the two oligonucleotides come in close proximity when they hybridise to adjacent sites on the nucleic acid being amplified, for example in the annealing phase of PCR, forming a fluorogenic complex. As more of the nucleic acid being amplified accumulates, so more of the fluorogenic complex can be formed and there is an increase in the fluorescence from the acceptor probe, and this can be measured. Hence the method allows detection of the amount of product as it is being formed. In another simple embodiment, and as applies to use of FRET probes in PCR based assays, one of the labelled oligonucleotides may also be a PCR primer used for PCR. In this configuration, the labelled PCR primer is part of the DNA strand to which the second labelled oligonucleotide hybridises, as described by Neoh et al (J Clin Path 1999;52:766-769.), von Ahsen et al (Clin Chem 2000;46:156-161), the entirety of which are encompassed by reference.

It will be appreciated by those of skill in the art that amplification and detection of amplification with hybridisation probes can be conducted in two separate phases-for example by carrying out PCR amplification first, and then adding hybridisation probes under such conditions as to measure the amount of nucleic acid which has been amplified. However, a preferred embodiment of the present invention utilises a combined PCR and hybridisation probing system so as to make the most of the closed tube or homogenous assay systems and is carried out on a Roche Lightcycler® or other similarly specified or appropriately configured instrument.

Such systems would also be adaptable to the detection methods described here. Those skilled in the art will appreciate that such probes can be used for allele discrimination if appropriately designed for the detection of point-mutations, in addition to deletion and insertions. Alternatively or in addition, the unlabelled PCR primers may be designed for allele discrimination by methods well known to those skilled in the art (Ausubel 1989-1999).

It will also be appreciated by those skilled in the art that detection of amplification in homogenous and/or closed tubes can be carried out using numerous means in the art, for example using TaqMan™ hybridisation probes in the PCR reaction and measurement of fluorescence specific for the target nucleic acids once sufficient amplification has taken place.

Although those skilled in the art will be aware that other similar quantitative “real-time” and homogenous nucleic acid amplification/detection systems exist such as those based on the TaqMan approach (U.S. Pat. Nos. 5,538,848 and 5,691,146), fluorescence polarisation assays (e.g. Gibson et al., 1997, Clin Chem., 43: 1336-1341), and the Invader assay (e.g. Agarwal et al., Diagn Mol Pathol 2000 September; 9(3): 158-164; Ryan D et al, Mol Diagn 1999 June; 4(2): 135-144). Such systems would also be adaptable to use the invention described, enabling real-time monitoring of nucleic acid amplification.

In one embodiment of the invention an initial procedure involves the manufacture of the oligonucleotide matrices or microchips. The microchips contain a selection of immobilized synthetic oligomers, said oligomers synthesized so as to contain complementary sequences for desired portions of transcription factor DNA. The oligomers are then hybridized with cloned or polymerase chain reaction (PCR) amplified transcription factor nucleic acids, said hybridization occurring under stringent conditions, outlined infra. The high stringency conditions insure that only perfect or near perfect matches between the sequence embedded in the microchip and the target sequence will occur during hybridization.

After each initial hybridization, the chip is washed to remove most mismatched fragments. The reaction mixture is then denatured to remove the bound DNA fragments, which are subsequently labelled with a fluorescent marker.

A second round of hybridization with the labelled DNA fragments is then carried out on sequence microchips containing a different set of immobilized oligonucleotides. These fragments first may be cleaved into smaller lengths. The different set of immobilized nucleotides may contain oligonucleotides needed for whole sequencing, partial sequencing, sequencing comparison, or sequence identification. Ultimately, the fluorescence from this second hybridization step can be detected by an epifluorescence microscope coupled to a CCD camera. (See U.S. Pat. No. 5,851,772 incorporated herein by reference).

Gene expression may alternatively be measured by immunological methods, such as immunohistochemical staining of tissue sections and assay of cell culture or body fluids, to quantitate directly the expression of the gene product, cytokine transcription factor. With immunohistochemical staining techniques, a cell sample is prepared, typically by dehydration and fixation, followed by reaction with labelled antibodies specific for the gene product coupled, where the labels are usually visually detectable, such as enzymatic labels, fluorescent labels, luminescent labels, and the like. A particularly sensitive staining technique suitable for use in the present invention is described by Hsu et al., 1980, Am. J. Clin. Path., 75:734-738. Antibodies useful for immunohistochemical staining and/or assay of sample fluids may be either monoclonal or polyclonal. Conveniently, the antibodies may be prepared against a synthetic peptide based on known DNA sequences of genes shown herein to be allergy-associated such as cig5, IFIT4, LAMP3, DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 or CISH comprises a sequence selected from the group consisting of sequences identified by probes 243610_at on human chromosome 9q21.13 at locus 138255, 1556097_at on human chromosome 15q25.2 and 242743_at on human chromosome 16p12.1 respectively.

For example, the allergy-associated gene peptides may be used as an immunogen to generate anti-cytokine transcription factor.antibodies. Such antibodies, which specifically bind to the products of the allergy-associated genes, are useful as standards in assays such as radioimmunoassay, enzyme-linked immunoassay, or competitive-type receptor binding assays radioreceptor assay, as well as in affinity purification techniques.

The invention will now be further described by way of reference only to the following non-limiting examples. It should be understood, however, that the examples following are illustrative only, and should not be taken in any way as a restriction on the generality of the invention described above. In particular, while the invention is described in detail in relation to the detection of mRNA for cig5, IFIT4, LAMP3, DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 and CISH comprises a sequence selected from the group consisting of sequences identified by probes 243610_at on human chromosome 9q21.13 at locus 138255, 1556097_at on human chromosome 15q25.2 and 242743_at on human chromosome 16p12.1 respectively, from HDM-exposed PMBC, it will be clearly understood that the findings herein are not limited to these specific allergens or methods.

### EXAMPLE 1

### USE OF MICROARRAY TO DETERMINE SPECIFIC

EXPRESSION OF mRNA IN ALLERGIC AND NON-ALLERGIC SUBJECTS IN RESPONSE TO ALLERGEN

Blood samples were obtained from 10 allergic individuals, who were selected on the basis of positive serum IgE responses to House Dust Mite (HDM), together with samples from 10 non-allergic controls who were tested for the presence of HDM-specific IgE in serum and were all negative. All 10 allergic subjects showed a wheal size of 5-14 mm in response to a skin prick test with allergen, whereas all non-allergic subjects showed a negative response. The presence of IgE directed against HDM was detected by the radioallergosorbent immunoassay capture test system, using the RAST (CAP) (Pharmacia, Australia), and the allergic volunteers in this study displayed RAST (CAP) scores ≧2.

Freshly-isolated peripheral blood mononuclear cells (PBMC) were resuspended at 1×10⁶ cells/ml, and 0.5 ml of the cell suspension was cultured for 16, 24 or 48 hours at 37° C., 5% CO₂ in round bottom plates in serum-free medium AIM-V⁴ (Life Technologies, Mulgrave, Australia) supplemented with 4×10⁻⁵ 2-mercaptoethanol, with or without the addition of 30 μg/mlof whole extract of HDM (Dermatophagoides pteronyssinus, CSL Limited, Parkville, Australia).

At each time point, equal-sized aliquots of cells were centrifuged and the cell pellets were used immediately for total RNA extraction. Alternatively, Dynabeads™ were used for positive selection of CD8⁺ T cells followed by positive selection of CD4⁺ T cells, prior to extraction of total RNA. Extraction of the RNA was performed by standard techniques. Total RNA was extracted using TRIZOL (Invitrogen) followed by an RNAeasy minikit (QIAGEN). Alternatively a Totally RNA extraction kit (Ambion, Austin, Tex., USA) could be used. If desired, messenger RNA can then be purified from 2 mg of total RNA using a MessageMaker™ kit (Invitrogen, The Netherlands).

The extracted RNA from the 10 individuals in each group (allergic and non-allergic) was pooled, and then labelled and hybridised to Affymetrix™ Genechip® U133A or U133plus2 arrays, using the standard Affymetrix™ protocols. Full details of the arrays and protocols are available on the Affymetrix website (http://www.affymetrix.com/index.affx). The U133A arrays provides probe sets corresponding to over 39,000 human genomes, while the U133plus2 array provides probe sets corresponding to over 47,000 transcripts, including all those from the U133A arrays. All of the corresponding nucleic acid sequences are available in publicly-available databases. Samples of the individual RNAs in the pools were kept separate for subsequent Taqman™ PCR validation studies (see Example 6 below).

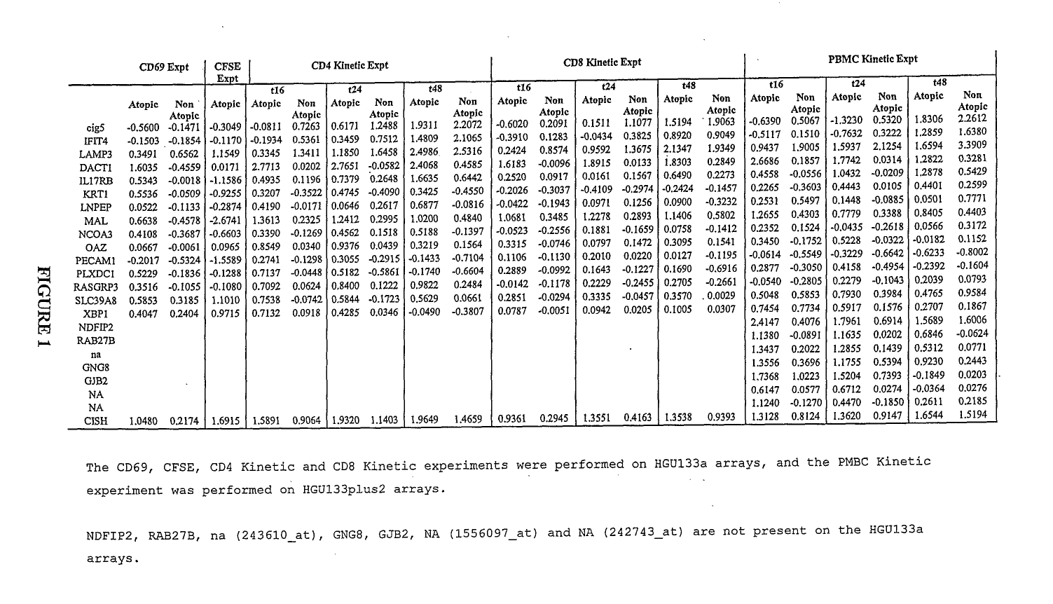

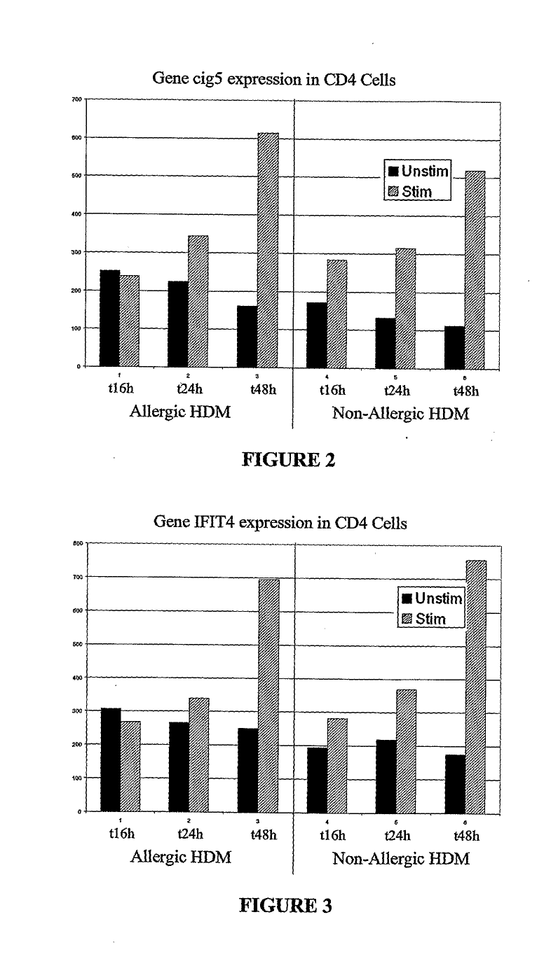

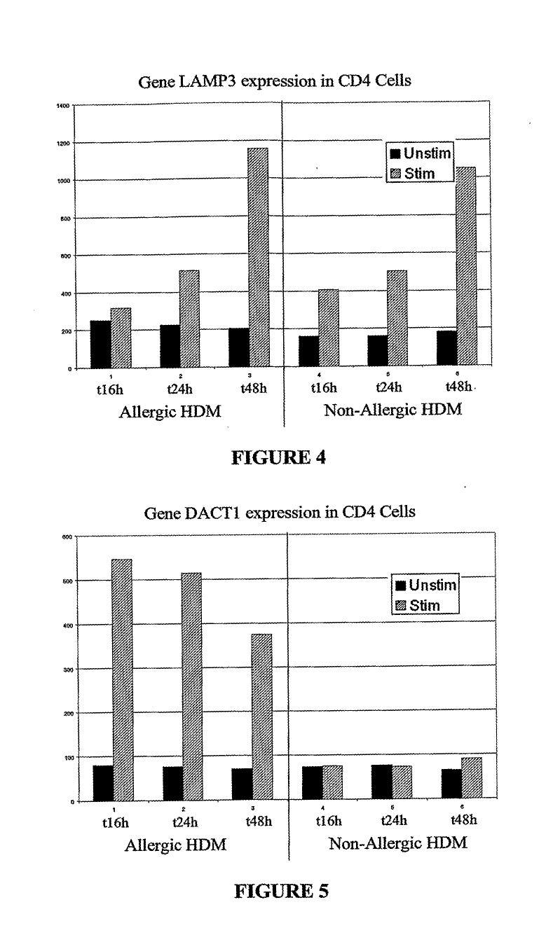

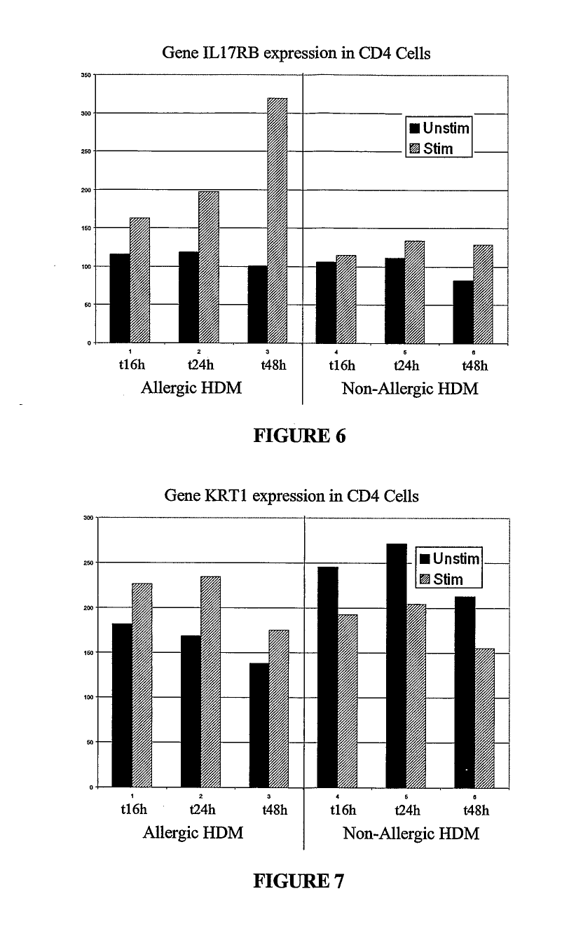

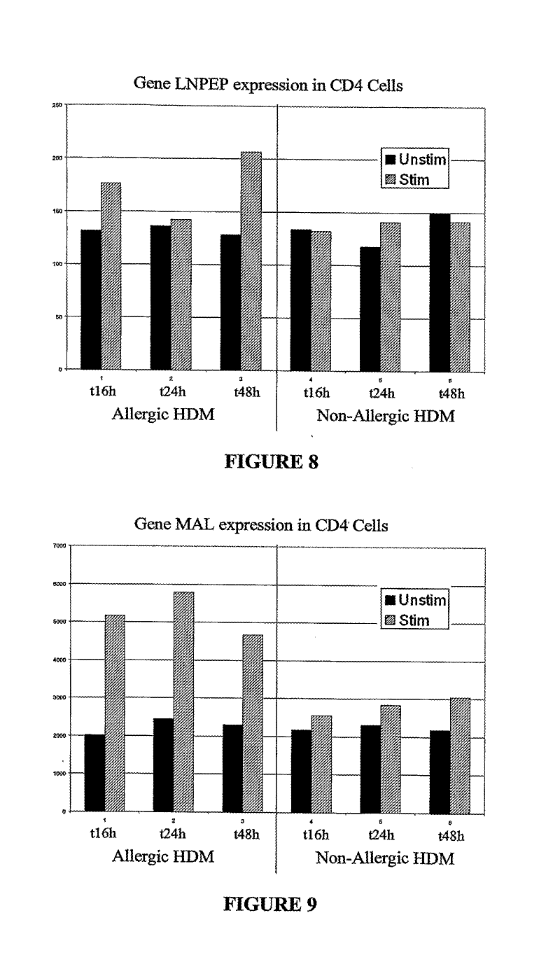

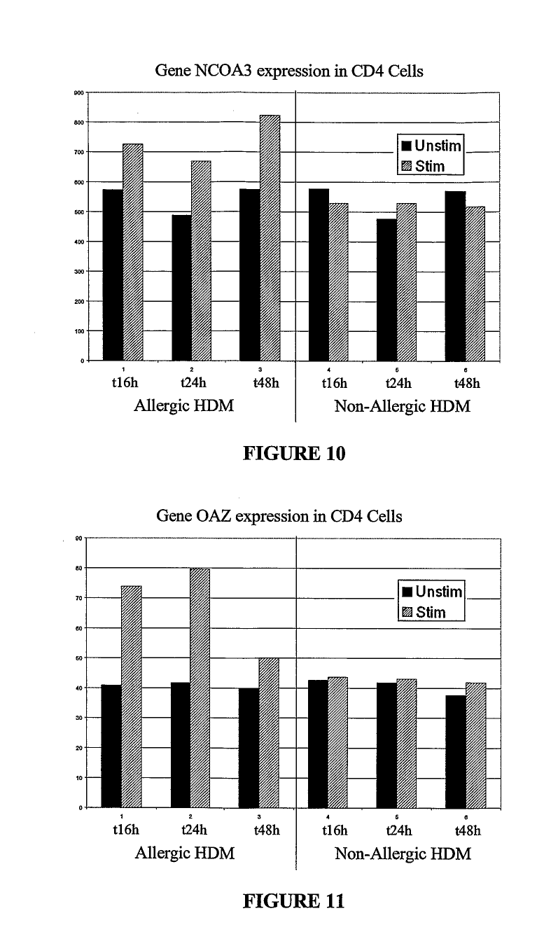

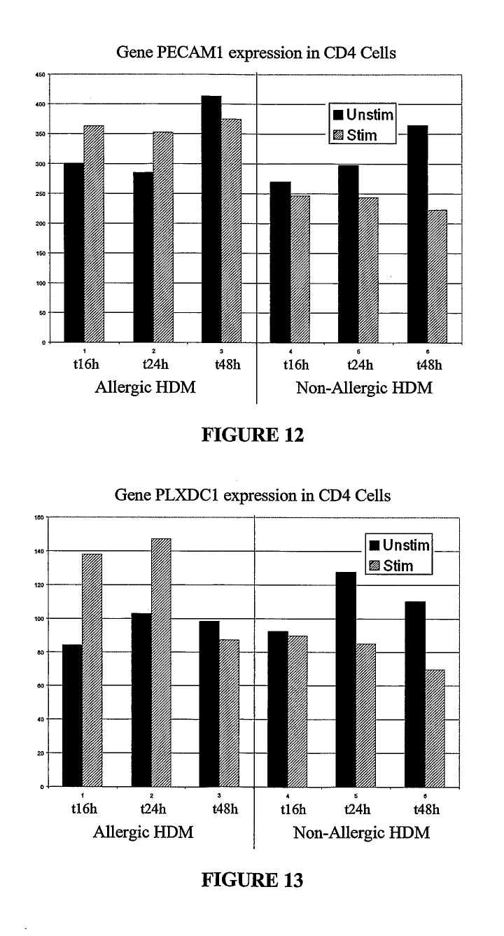

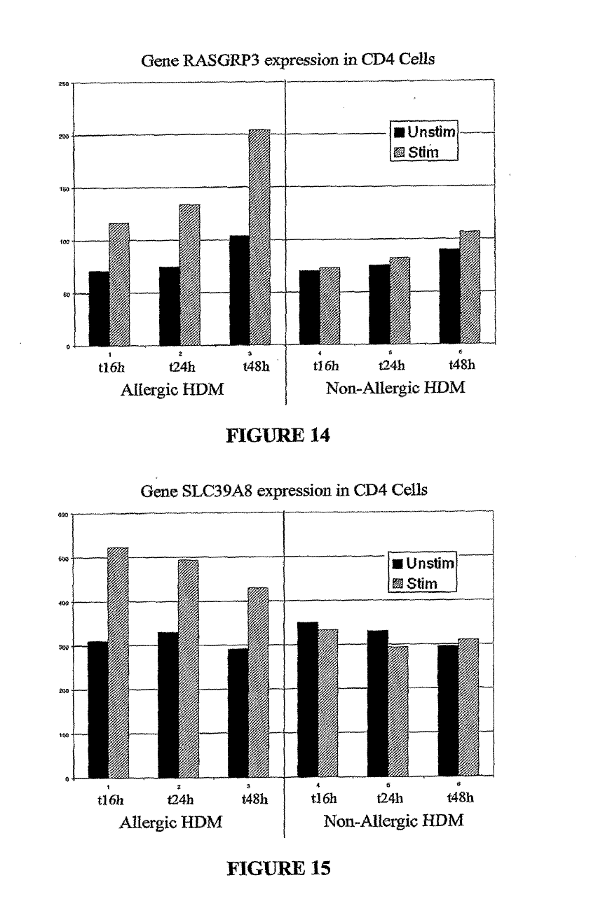

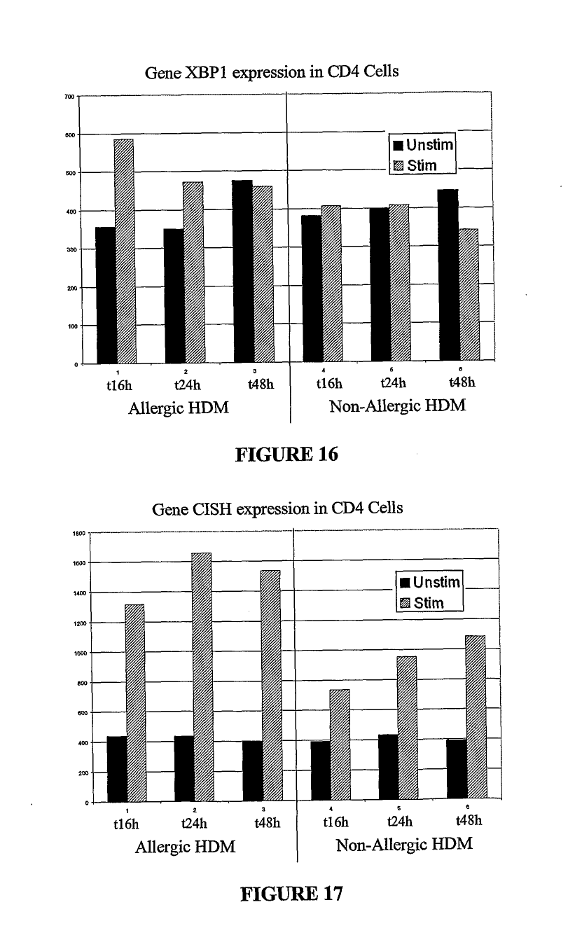

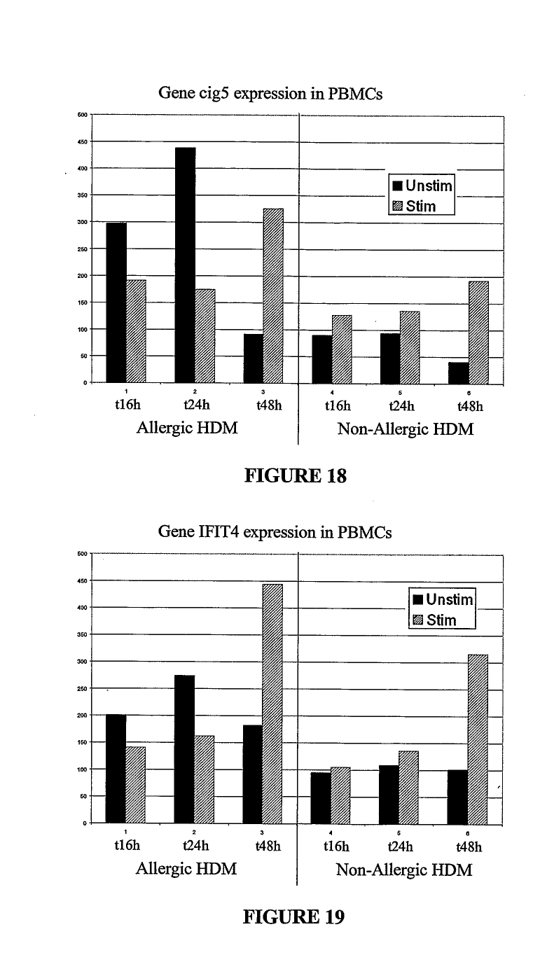

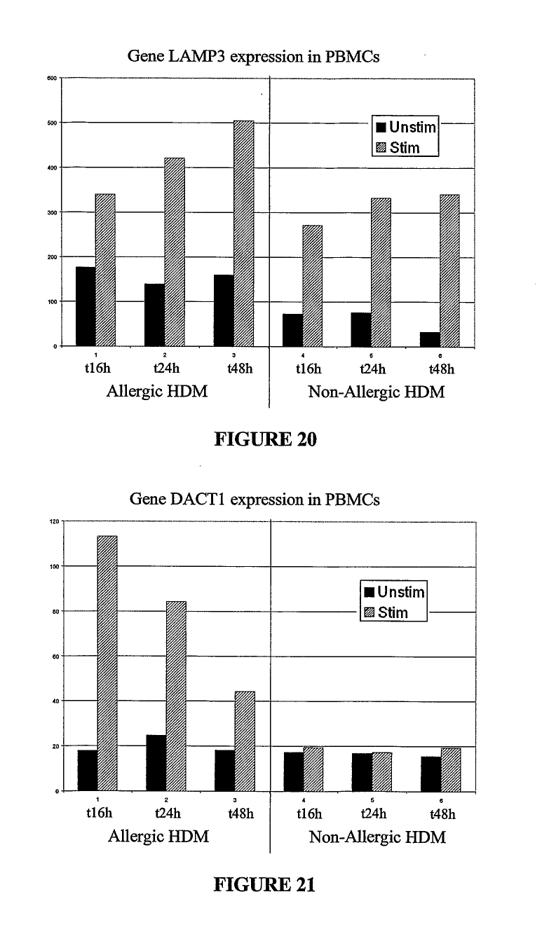

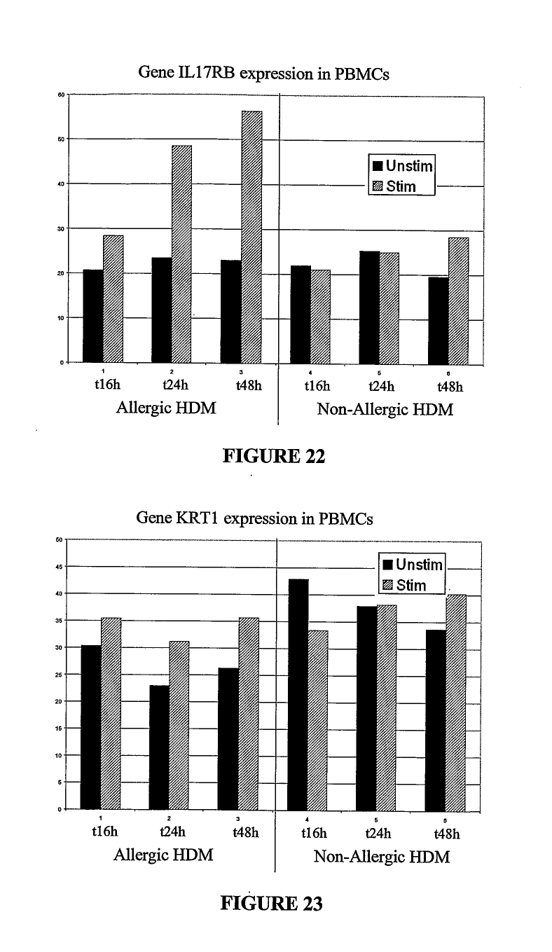

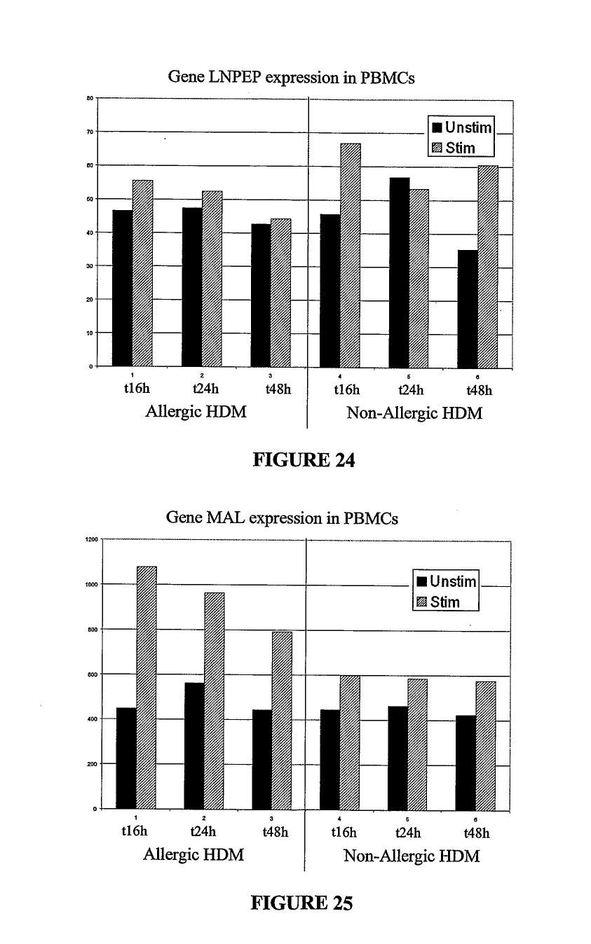

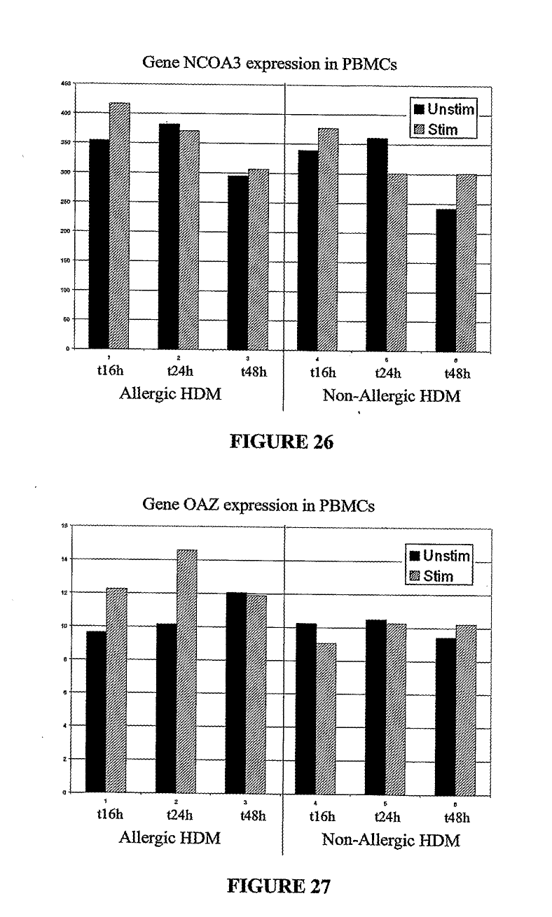

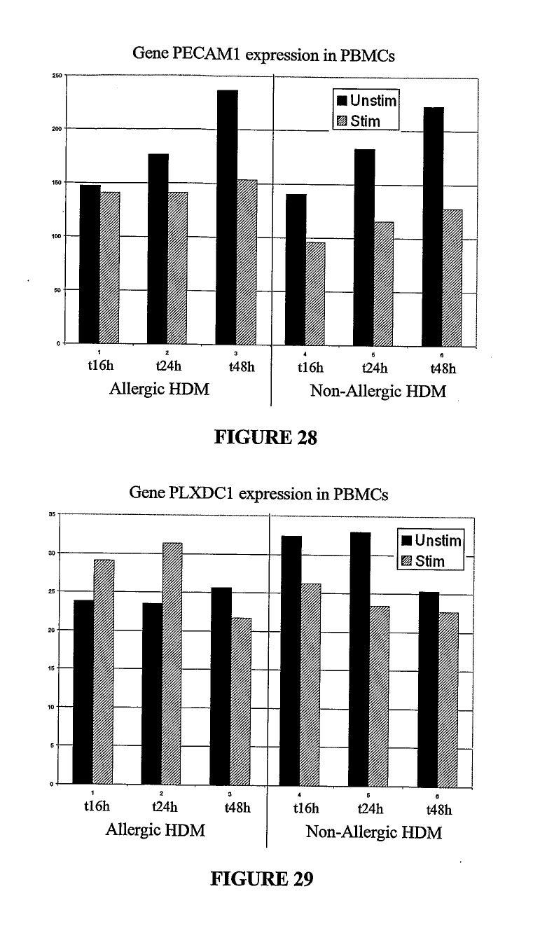

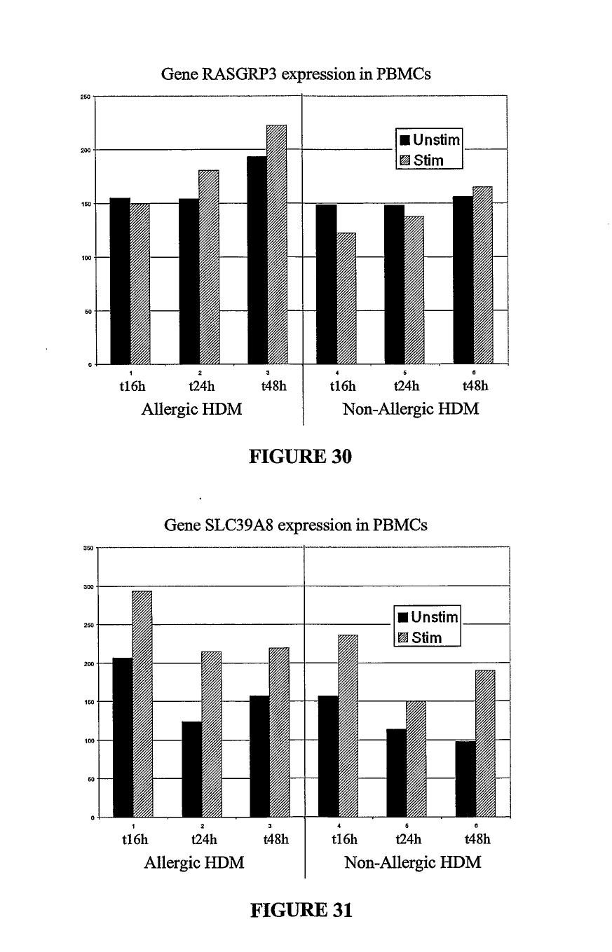

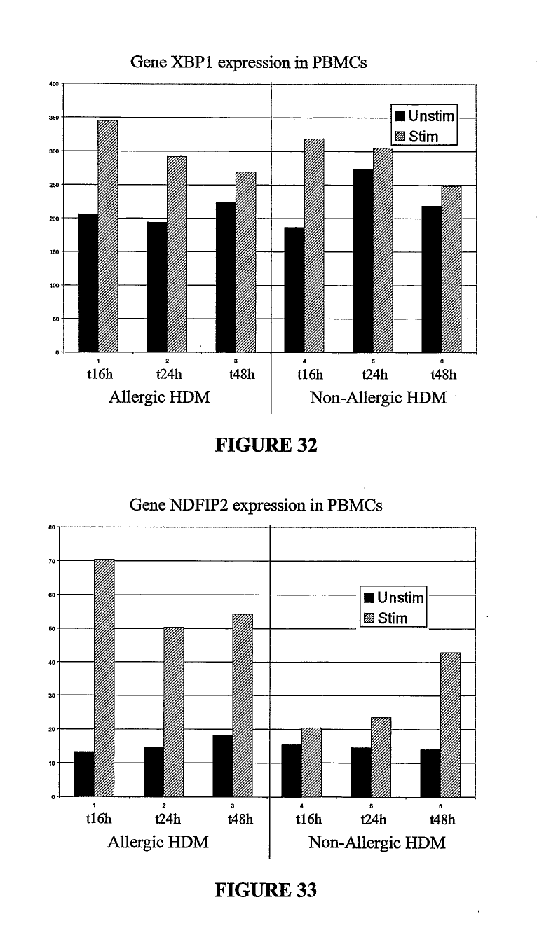

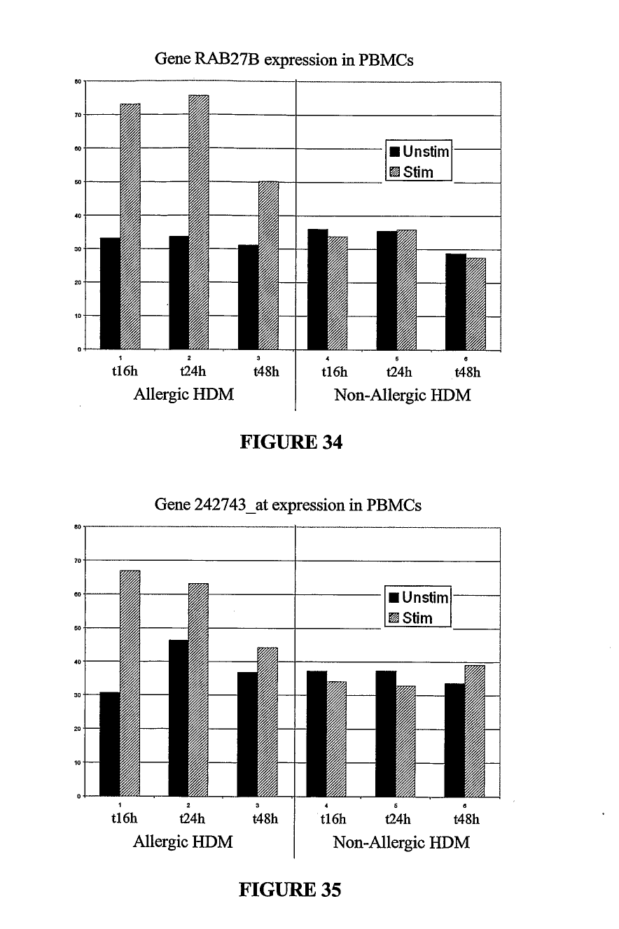

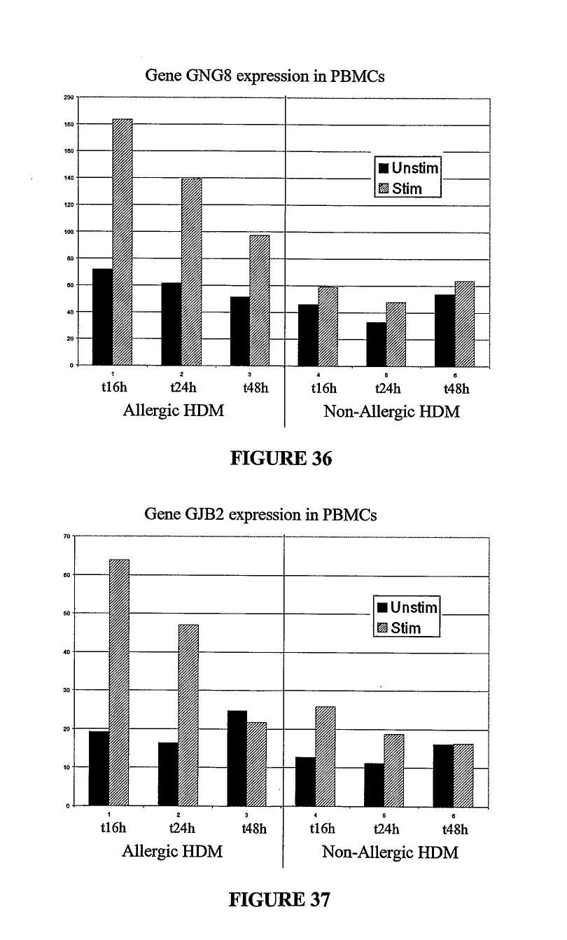

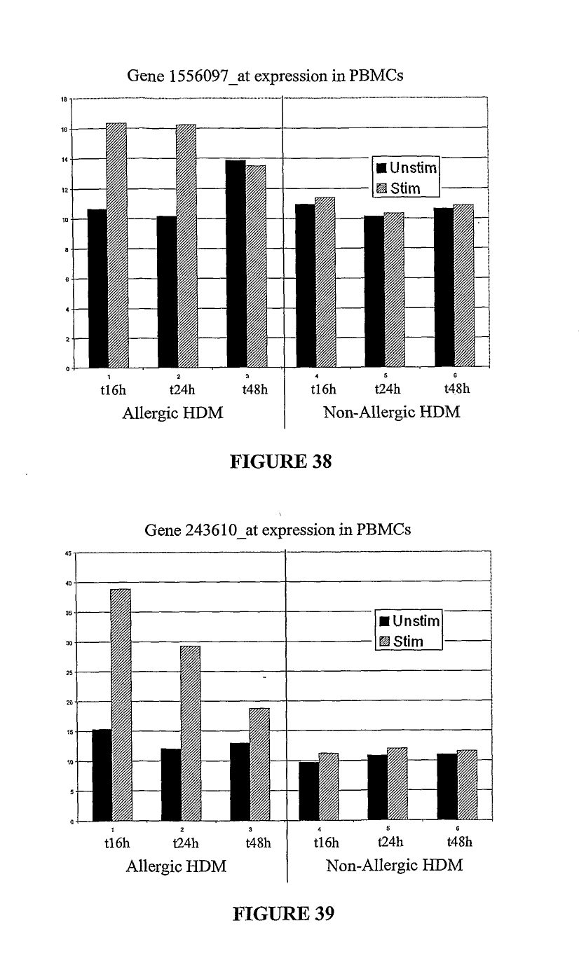

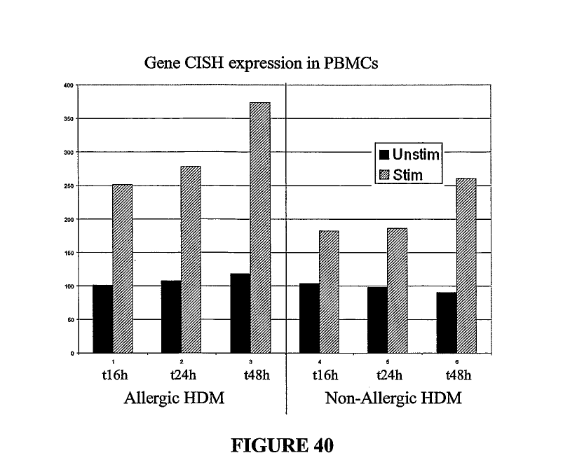

Data from these microarray experiments, and those of Examples 2-3 below, are shown in FIG. 1 as fold expression (stimulated vs unstimulated cultures) on a log 2 scale. Data were analysed with the rma algorithm using the statistical package R (Irizarry R. A. et al. 2003, Biostatistics 4(2):249-64). Genes were considered to be differentially expressed in stimulated and unstimulated cultures if the fold-change value was greater than the cut-off value (background noise), which is shown for each experiment. Cut-off values were determined on the basis of the standard deviation of the noise for each experiment. Genes which were consistently expressed at different levels in samples from allergic and non-allergic individuals, i.e. genes with large fold-change values between allergic individuals and non-allergic individuals were then identified. A total of 23 genes showed differential expression; of these 16 were identified using the U133a arrays, and a further 7 were identified using the U133plus2 arrays. Illustrative expression patterns of the selected genes are shown in FIGS. 2 to 40. Data in these figures are shown as absolute expression intensity levels on a linear scale.

The microarray data summarized in FIGS. 2 to 40 shows that cig5, IFIT4 and LAMP3 are upregulated in non-allergic individuals, i.e. these genes are upregulated in HDM-stimulated cultures compared to unstimulated cultures to a greater extent in the non-allergic individuals than the allergic individuals, at least at 16 and 24 hours of culture. The remaining 20 genes are upregulated in the allergic individuals, and indeed KRT1, PECAM1 and PLXDC1 are actually down regulated in the non-allergic individuals.

These data were interpreted as follows: If the genes are “protective” i.e. if the expression of these genes is associated with absence of allergy, then allergic individuals will have expression levels which are lower than those non-allergic individuals. For example, in the PBMC kinetic experiment at 48 hours post-stimulation cig5 shows a figure of 1.8306 for allergic individuals, which is lower than the corresponding figure of 2.2612 for non-allergic individuals. Similarly, IFIT4 shows a figure of 1.2859 for allergic individuals, which is lower than the corresponding figure of 1.6380 for non-allergic individuals. The expression of these genes is therefore considered to be “protective”.

Genes which are indicative of allergic disorder are those in which the expression level for allergic individuals is higher than that for non-allergic individuals. For example, DACT1 in the PBMC kinetic experiment at 48 days post-stimulation shows a figure of 1.2822 for allergic individuals, which is higher than the corresponding figure for non-allergic individuals at 0.3281. Similarly, IL17RB shows a figure of 1.2878 for allergic individuals, which is higher than the corresponding figure for non-allergic individuals at 0.5429. The expression of these genes is therefore considered to be “predictive of a predisposition for allergy”.

### EXAMPLE 2

### USE OF MICROARRAY ANALYSIS TO DETERMINE

SPECIFIC EXPRESSION OF mRNA ISOLATED FROM CD69⁺ CELLS FROM ALLERGIC AND NON-ALLERGIC SUBJECTS

Blood samples were obtained from 4 allergic individuals, who were selected on the basis of positive serum IgE responses to House Dust Mite (HDM), and from 4 non-allergic controls who were tested for the presence of HDM-specific IgE in serum and were all negative. The presence of IgE directed against HDM was detected by the RAST. (CAP) system (Pharmacia, Australia), and the allergic volunteers in this study displayed RAST (CAP) scores ≧2.

PBMCs from all individuals were cultured in the presence or absence of HDM (10 μg/ml) for 14 hours, as described in Example 1. Following the 14 hour stimulation, monocytes and B cells, which express high levels of CD69, were removed using Dynabeads™ coated with CD14 and CD19 in accordance with the manufacturer's instructions. Activated CD69⁺ T cells were then positively selected from the remaining cell population, using Dynabeads™ coated with anti-CD69 monoclonal antibody. RNA was extracted, labelled and hybridised to Affymetrix™ U133a arrays using the standard Affymetrix™ protocols, as described in Example 1.

The results of these experiments are shown in FIG. 1 column 1, and the data analysis was performed as described in Example 1. Again it can be seen that for those genes which are considered “protective”, allergic individuals had lower levels of expression relative to non-allergic individuals, while those genes considered to be predictive of a predisposition to develop allergy were more highly expressed in allergic individuals than in non-allergic individuals. It is worthy of note that the closer the figures are to zero, the greater the level of interference from the background and therefore the less accurate the test.

### EXAMPLE 3

### USE OF MICROARRAY ANALYSIS TO DETERMINE SPECIFIC EXPRESSION OF mRNA ISOLATED FROM RECENTLY-DIVIDED CELLS FROM ALLERGIC AND NON-ALLERGIC SUBJECTS

PBMCs from 4 allergic and 4 non-allergic individuals were labelled with 5 μm carboxy-fluorescein diacetate, succinimidyl ester (CFSE) by standard procedures, and then stimulated with HDM (10 μg/ml) for 6 days as described in Example 1. The CFSE fluorescence stain is used to monitor cell division. Cells which are the progeny of recent cell division events show a low degree of staining (CFSE^{low}); non-dividing cells are strongly stained. Live progeny cells (CFSE^{low}) were sorted by flow cytometry, rested overnight, and then stimulated with PMA and ionomycin for 6 hours. RNA was extracted, labelled and hybridised to Affymetrix™ U133a arrays using the standard Affymetrix™ protocols as described above.

The results of these experiments are shown in FIG. 1 column 2, and the data analysis was performed as described in Example 1. In these experiments there was very poor proliferation of HDM-specific cells from non-allergic subjects, and so sufficient RNA to analyse only the allergic subjects was obtained. However, this experiment does demonstrate that it is possible to concentrate specific T-cells by initial generation of a “cell line” which is then re-stimulated using agents known in the art to provide “optimal” stimulation e.g. PMA/ionomycin. This is in contrast to the more subtle stimulus of specific allergen at low concentration applied to unfractionated T-cells using total PBMC.

### EXAMPLE 4

### VALIDATION OF RESULTS FROM EXAMPLE 1

IL-4 is the essential growth factor for all Th2 cells. Therefore to confirm the “Th2 status” of each PBMC sample, real-time quantitative PCR was performed in order to measure expression levels of the index gene IL-4 in RNA extracts from 48 hr cell pellets from the individual samples used to generate the pools for the kinetic experiment described in Example 1, using ABI Prism 7900HT Sequence Detection System.

Standard PCR premixes were prepared using QuantiTect SYBRGreen PCR Master Mix (QIAGEN), containing 2.5 mM MgCl₂ (final concentration). SYBRGreen binds to all double-stranded DNA, so no probe is needed. Primers were used at a final concentration of 0.3 μM. Standard conditions were used, except that 15 minutes instead of 10 minutes was used for HotStar Taq polymerase activation. In addition, a dissociation step was included and melt curve analysis performed to confirm amplification of a single product. Amplified products have been or will be sequenced to confirm specific amplification of the target of interest.

The primers used for the PCR were:

| | |
|---|---|
| IL-4 Forward: | |
| 5′AACAGCCTCACAGAGCAGAAGACT3′ | (SEQ ID NO:47) |
| IL4 Reverse: | |
| 5′CAGCGAGTGTCCTTCTCATGGT3′ | (SEQ ID NO:48) |
| LAMP3 forward: | |
| 5′GCGTCCCTGGCCGTAATTT3′ | (SEQ ID NO:5) |
| LAMP3 reverse: | |
| 5′TGGTTGCTTAGCTGGTTGCT3′ | (SEQ ID NO:6) |
| DACT1 forward: | |
| 5′AACTCGGTGTTCAGTGAGTGT3′ | (SEQ ID NO:7) |
| DACT1 reverse: | |
| 5′GGAGAGGGAACGGCAAACT3′ | (SEQ ID NO:8) |
| PLXDC1 forward: | |
| 5′CCTGGGCATGTGTCAGAGC3′ | (SEQ ID NO:23) |
| PLXDC1 reverse: | |
| 5′GGTGTTGGAGAGTATTGTGTGG3′ | (SEQ ID NO:24) |
| cig5 forward: | |
| 5′CAAGACCGGGGAGAATACCTG3′ | (SEQ ID NO:1) |
| cig5 reverse: | |
| 5′GCGAGAATGTCCAAATACTCACC3′ | (SEQ ID NO:2) |
| IFIT4 forward: | |
| 5′GAGTGAGGTCACCAAGAATTC3′ | (SEQ ID NO:3) |
| IFIT4 reverse: | |
| 5′CACTCTATCTTCTAGATCCCTTGAGA3′ | (SEQ ID NO:4) |
| MAL forward: | |
| 5′TCGTGGGTGCTGTGTTTACTCT3′ | (SEQ ID NO:15) |
| MAL reverse: | |
| 5′CAGTTGGAGGTTAGACACAGCAA3′ | (SEQ ID NO:16) |
| NCOA3 forward: | |
| 5′CCTGTCTCAGCCACGAGCTA3′ | (SEQ ID NO:17) |
| NCOA3 reverse: | |
| 5′TCCTGAAAGATCATGTCTGGTAA3′ | (SEQ ID NO:18) |
| PECAM1 forward: | |
| 5′AGTCCAGATAGTCGTATGTGAAATGC3′ | (SEQ ID NO:21) |
| PECAM1 reverse: | |
| GGTCTGTCCTTTTATGACCTCAAAC3′ | (SEQ ID NO:22) |
| SLC39A8 forward: | |
| 5′GCAGTCTTACAGCAATTGAACTTT3′ | (SEQ ID NO:27) |
| SLC39A8 reverse: | |
| 5′CCATATCCCCAAACTTCTGAA3′ | (SEQ ID NO:28) |
| XBP1 forward: | |
| 5′GTAGATTTAGAAGAAGAGAACCAAAAAC3′ | (SEQ ID NO:29) |
| XBP1 reverse: | |
| 5′CCCAAGCGCTGTCTTAACTC3′ | (SEQ ID NO:30) |
| NDFIP2 forward: | |
| 5′AGTGGGGAATGATGGCATTTT3′ | (SEQ ID NO:31) |
| NDFIP2 reverse: | |
| AAATCCGCAGATAGCACCA3′ | (SEQ ID NO:32) |
| RAB27B forward: | |
| 5′CAGAAACTGGATGAGCCAACT3′ | (SEQ ID NO:33) |
| RAB27B reverse: | |
| 5′GACTTCCCTCTGATCTGGTAGG3′ | (SEQ ID NO:34) |
| 243610_at forward: | |
| 5′TGCATTGACAACGTACTCAGAA3′ | (SEQ ID NO:35) |
| 243610_at reverse: | |
| 5′TCATCTTGACAGGGATAAGCAT3′ | (SEQ ID NO:36) |
| GNG8 forward: | |
| 5′GAACATCGACCGCATGAAGGT3′ | (SEQ ID NO:37) |
| GNG8 reverse: | |
| 5′AGAACACAAAAGAGGCGCTTG3′ | (SEQ ID NO:38) |
| GJB2 forward: | |
| 5′GCTTCCTCCCGACGCAGA3′ | (SEQ ID NO:39) |
| GJB2 reverse: | |
| 5′AACGAGGATCATAATGCGAAA3′ | (SEQ ID NO:40) |
| 1556097_at forward: | |
| 5′TCTTATTTCACTTTCTCAACTCATCA3′ | (SEQ ID NO:41) |
| 1556097_at reverse: | |
| 5′GGCATAACCTGAATGTATAATTCAA3′ | (SEQ ID NO:42) |
| 242743_at forward: | |
| 5′GAAAAAGCTGTTGAGTGAAGAAGACT3′ | (SEQ ID NO:43) |
| 242743_at reverse: | |
| 5′TGCAGGATGAGCAATGCTGAGA3′ | (SEQ ID NO:44) |
| and | |
| CISH forward: | |
| 5′GGGAATCTGGCTGGTATTGG3′ | (SEQ ID NO:45) |
| CISH reverse: | |
| 5′TTCTGGCATCTTCTGCAGGTGTT3′. | (SEQ ID NO:46) |

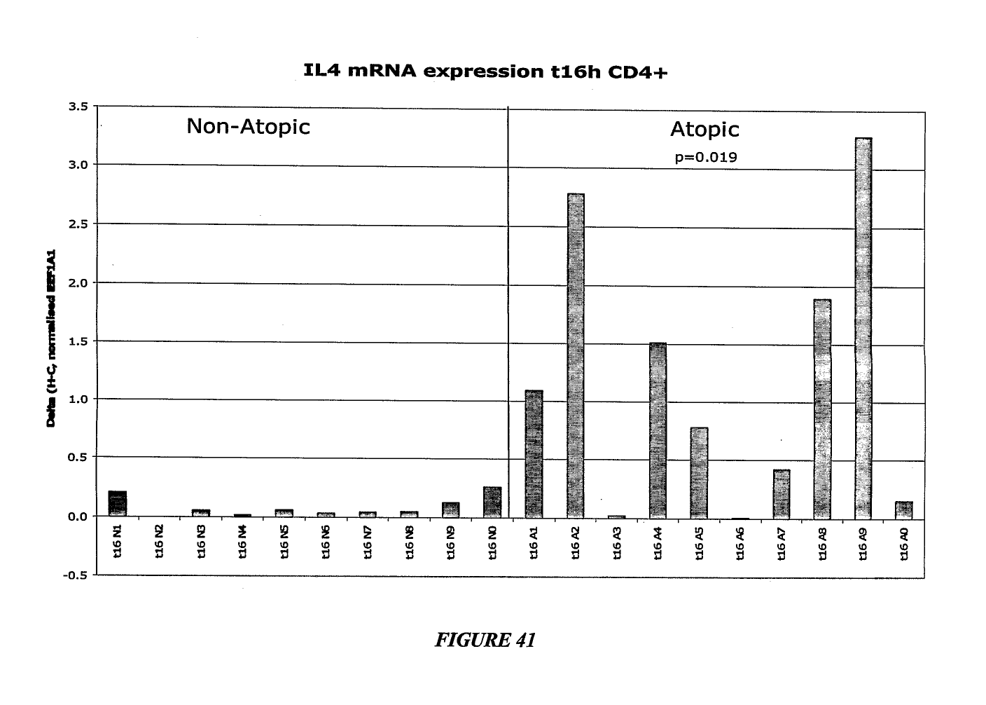

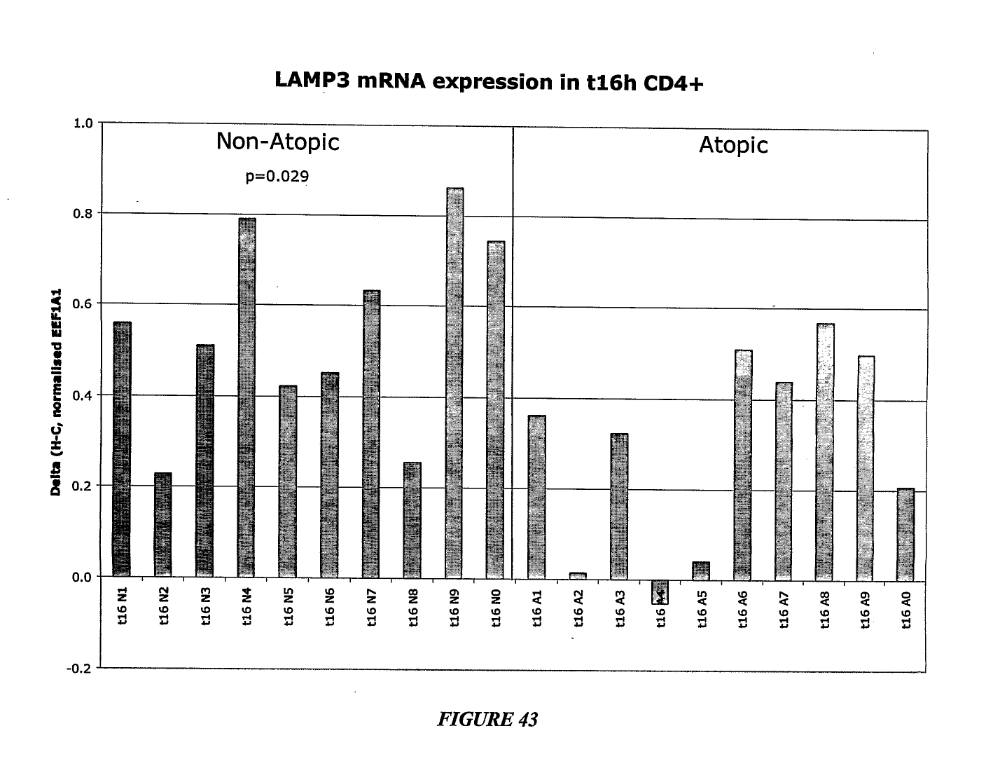

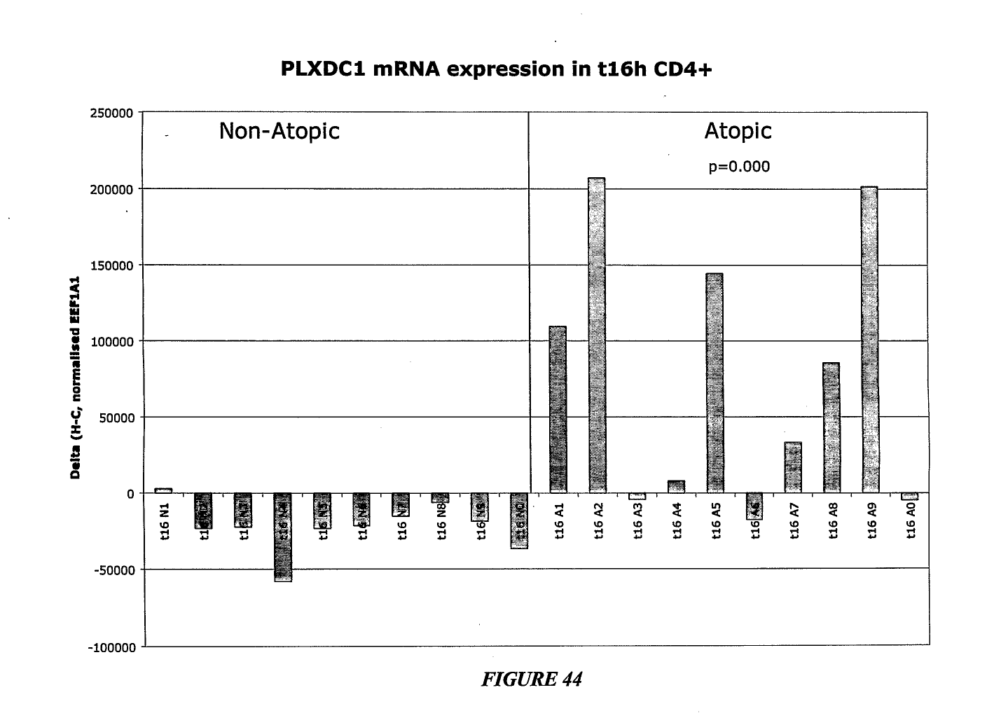

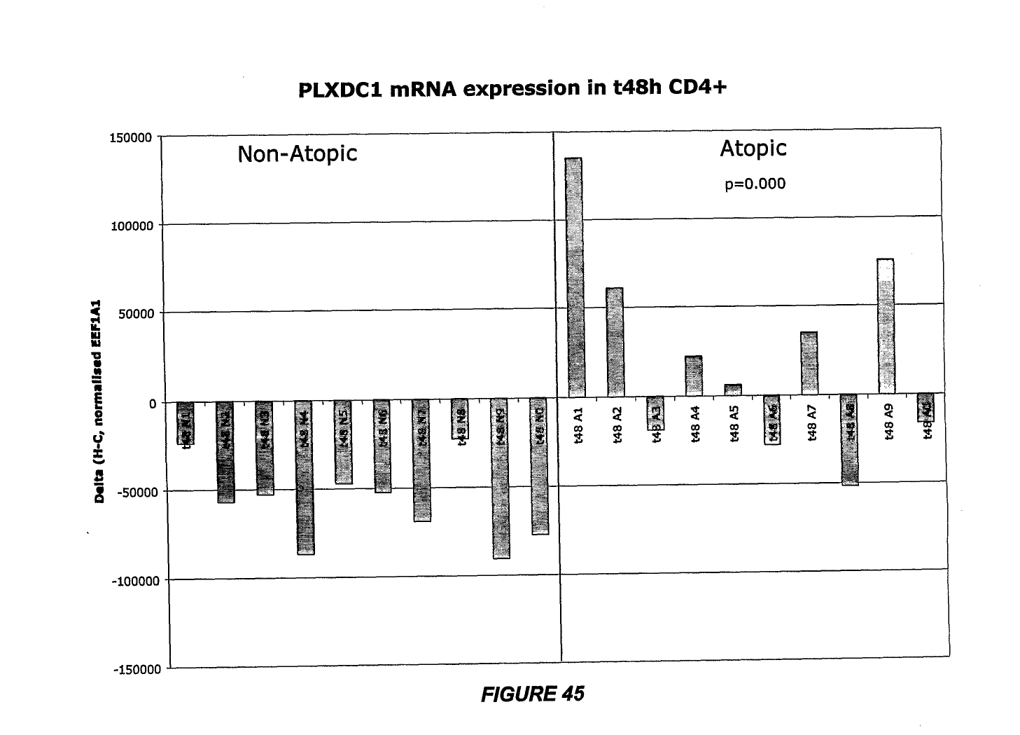

The data were normalised to the EEF1A1 housekeeping gene, and the results are shown in FIGS. 41 to 45. It can be seen from FIG. 41 that 7 of the samples from putative allergic individuals initially selected for the culture experiment displayed vigorous IL-4 gene transcription, while 3 samples, indicated by arrows, showed low/undetectable responses. This indicated that, despite their positive skin-prick test status, the numbers of HDM-specific T-cells present in the circulation of these individuals at the time of sample were too low for detection of in vitro immune responses. These 3 subjects showed the lowest response in the skin prick test (5-6 mm), and also showed a low level of expression of DACT1 and PLXDC1, but a high level of expression of LAMP3.

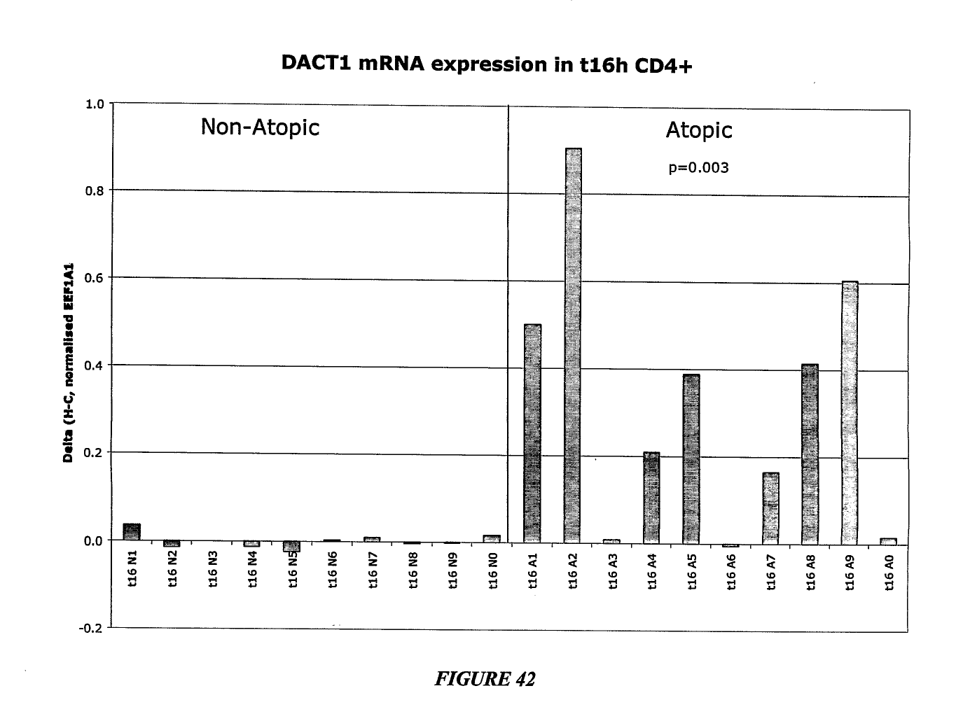

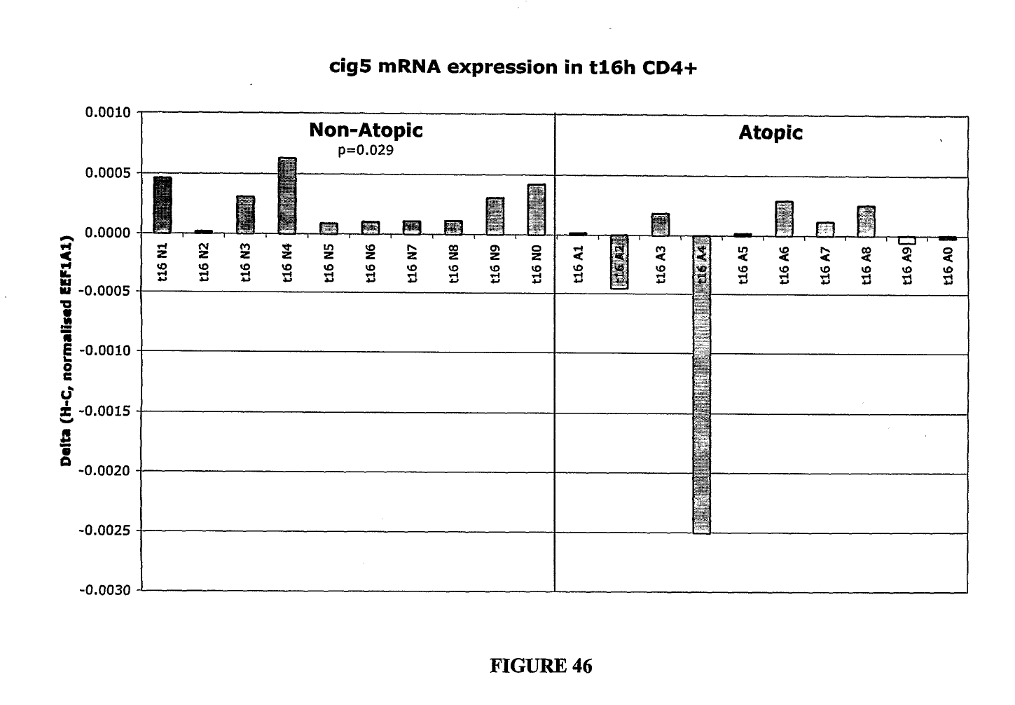

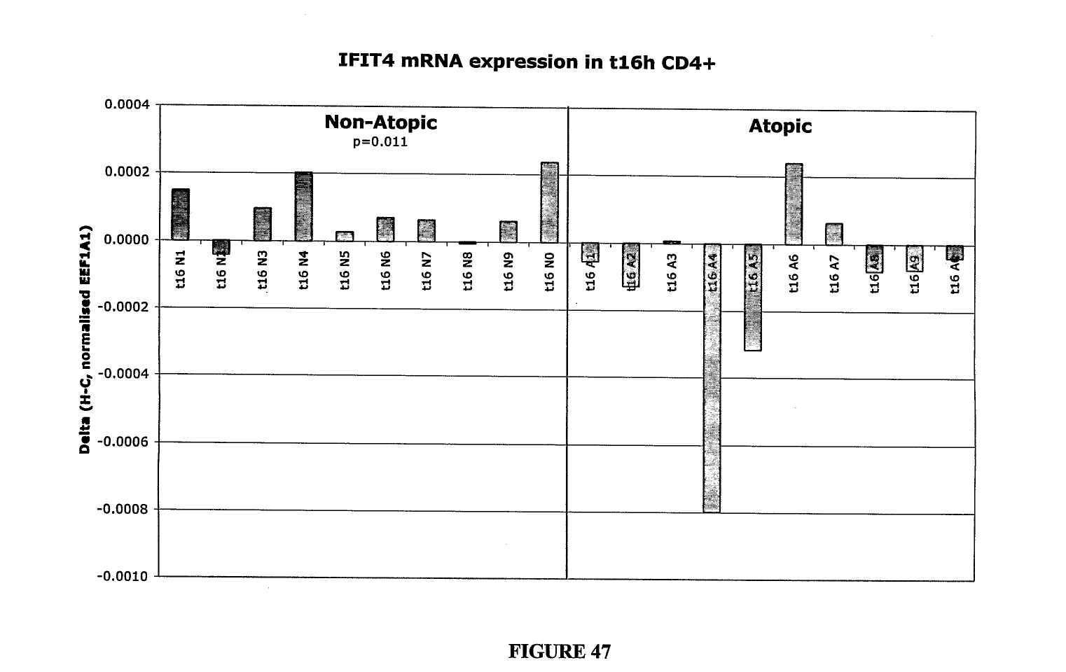

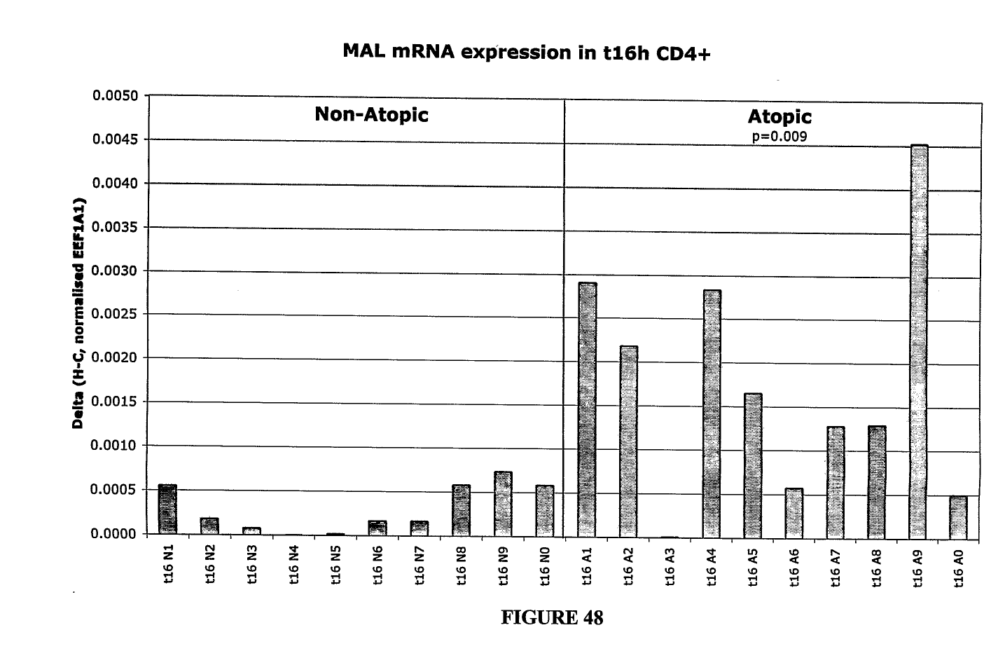

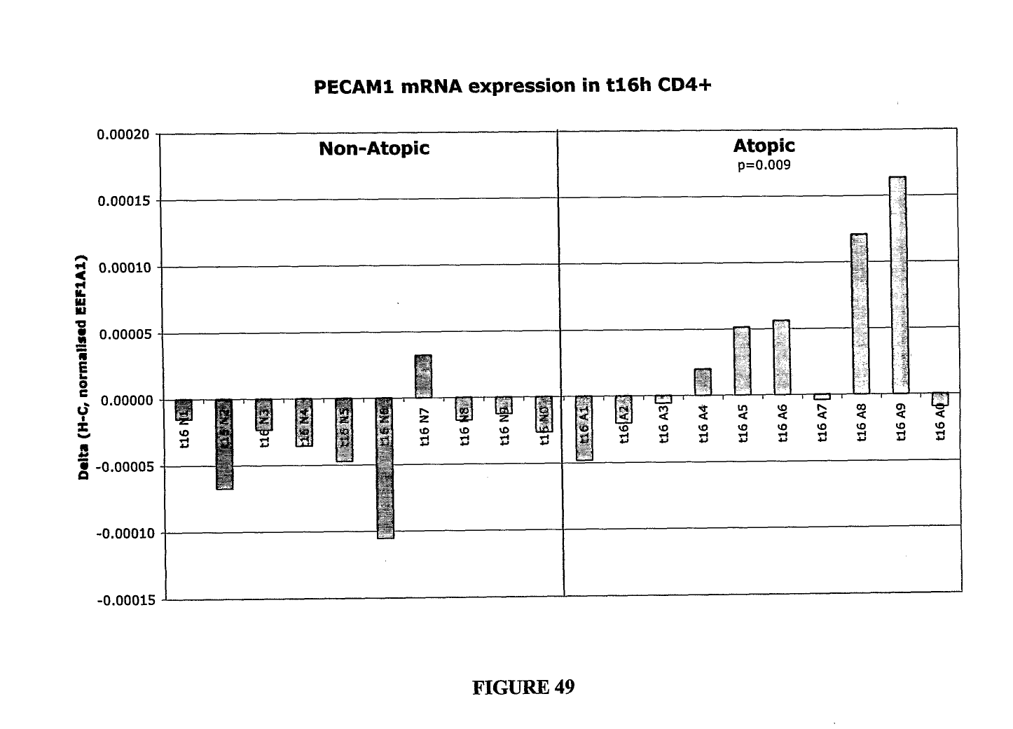

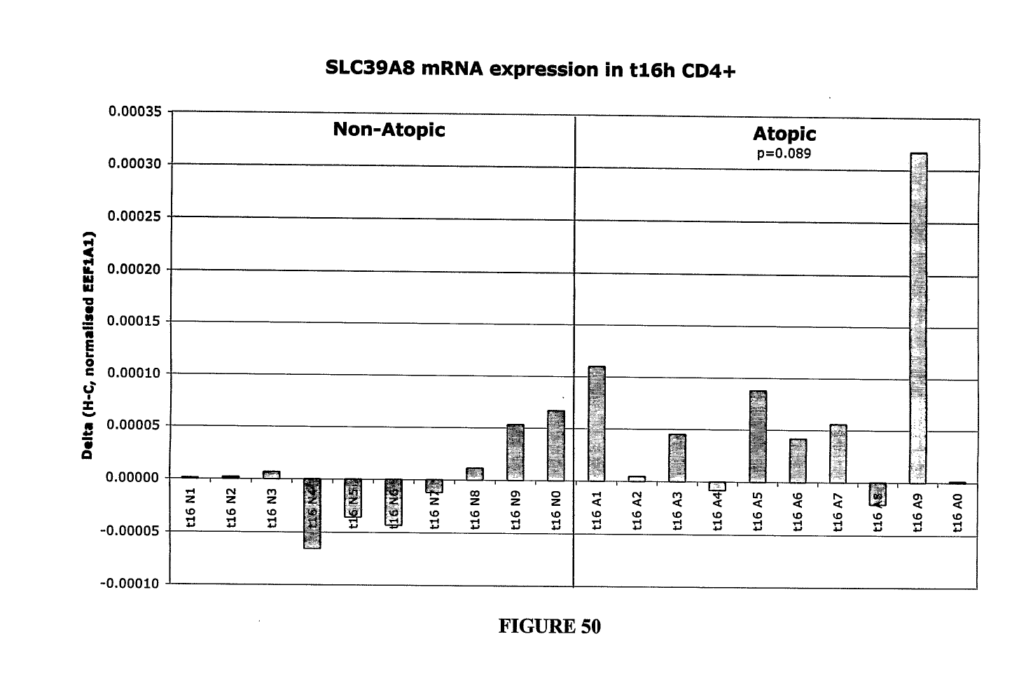

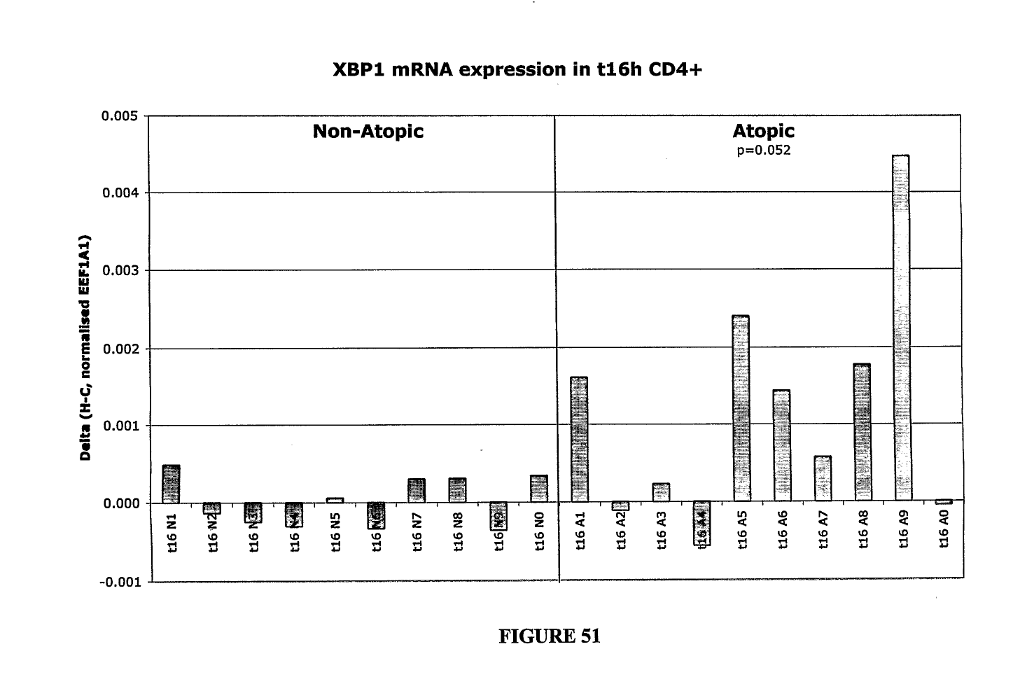

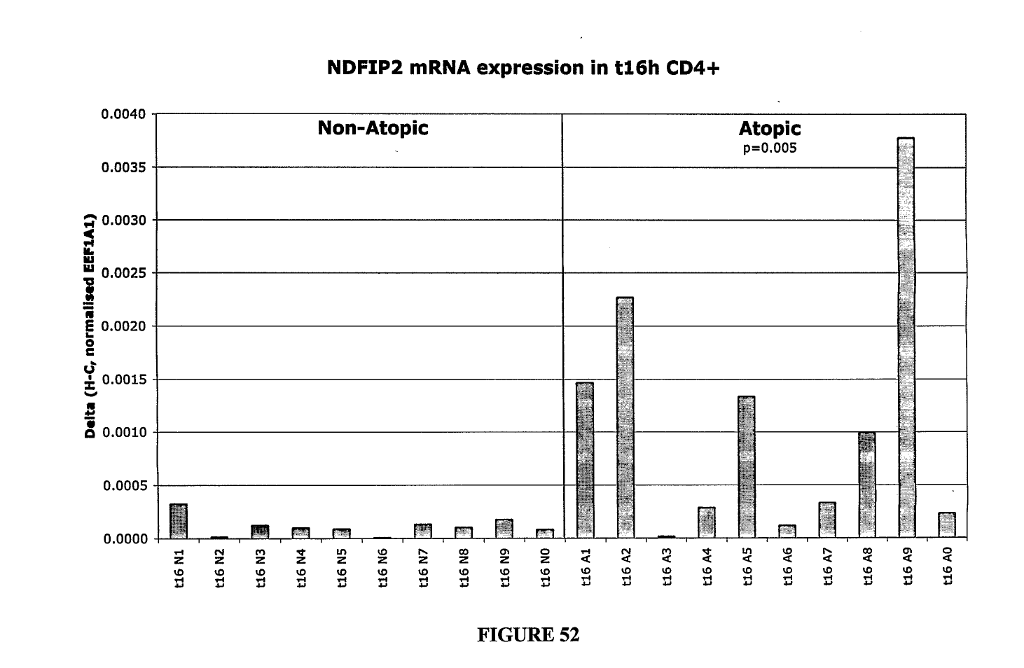

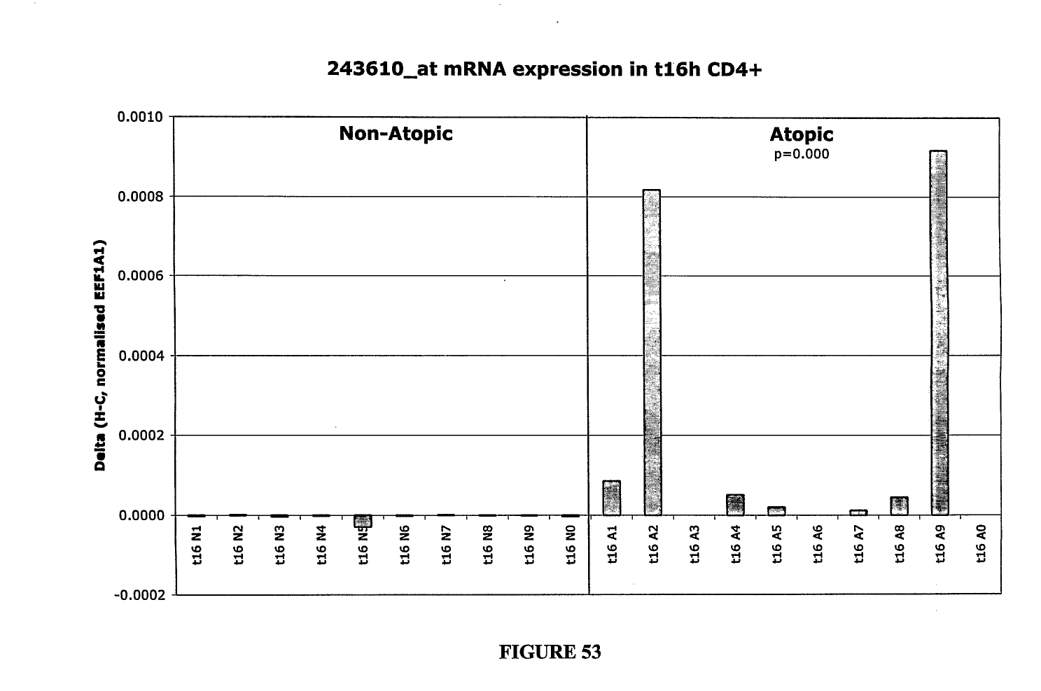

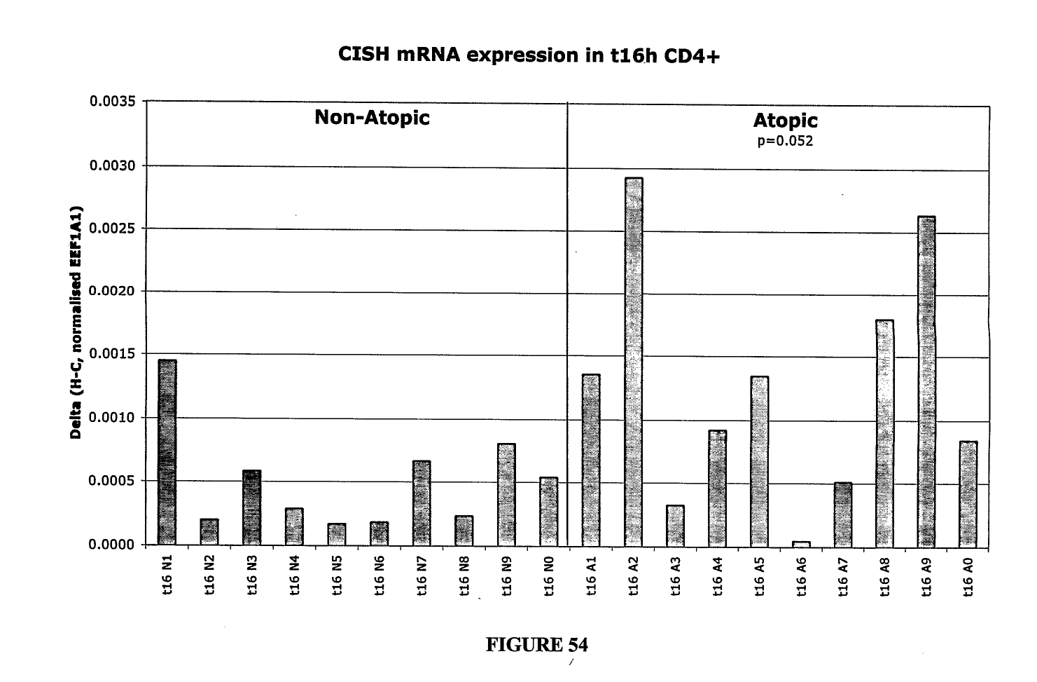

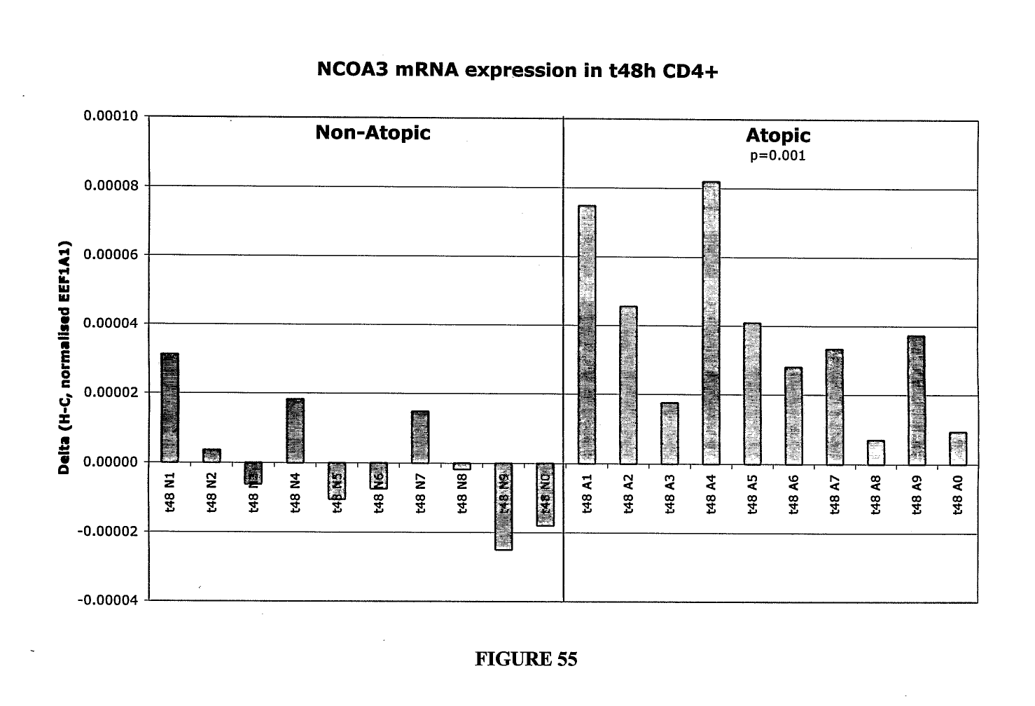

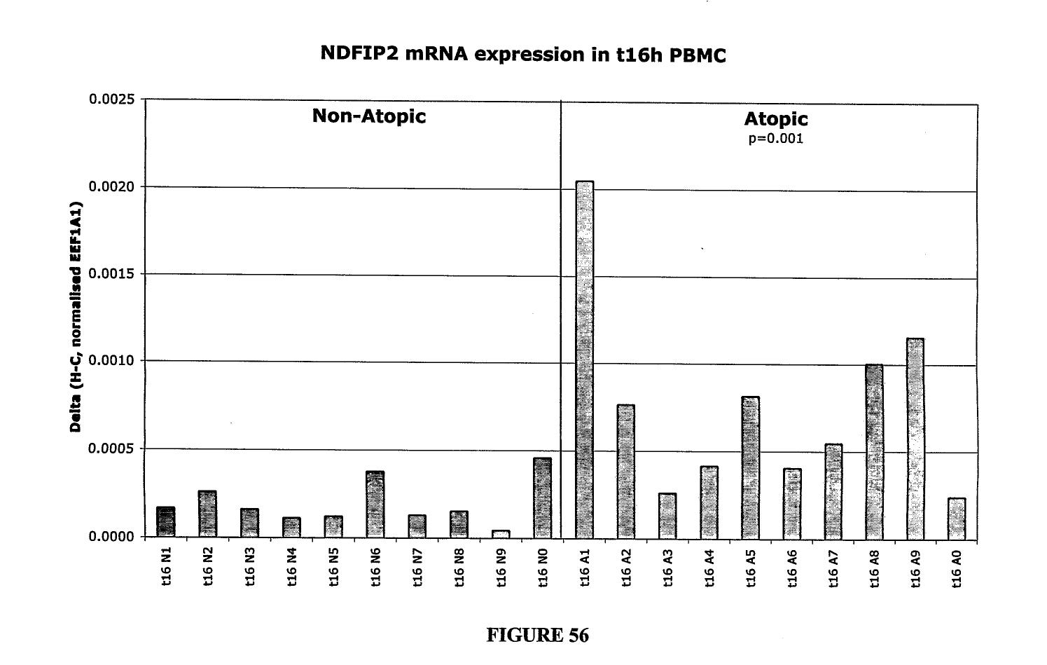

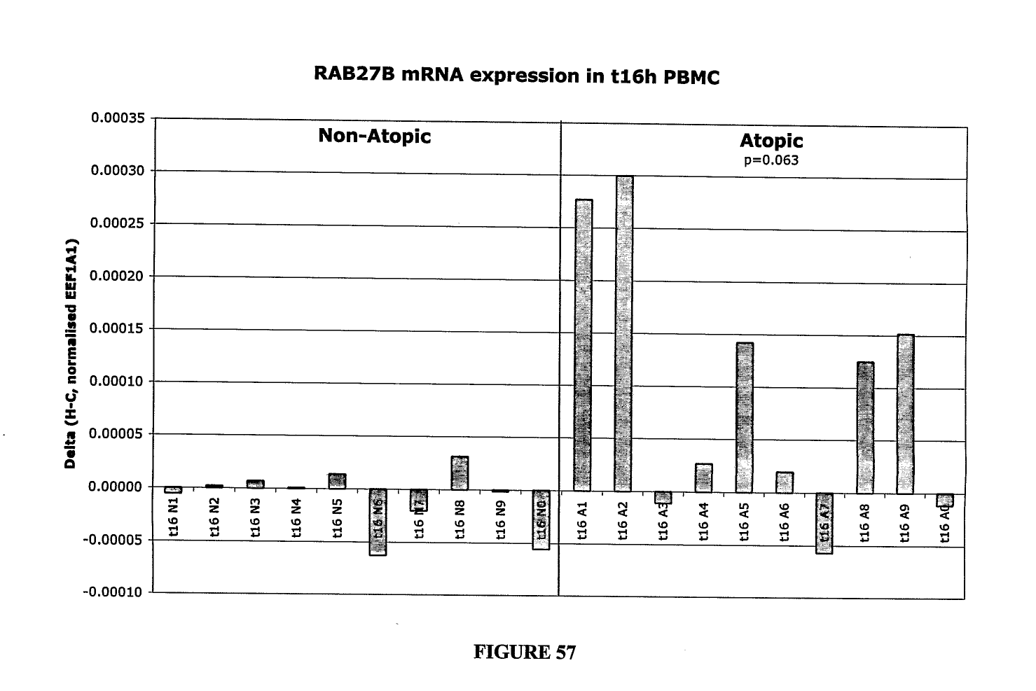

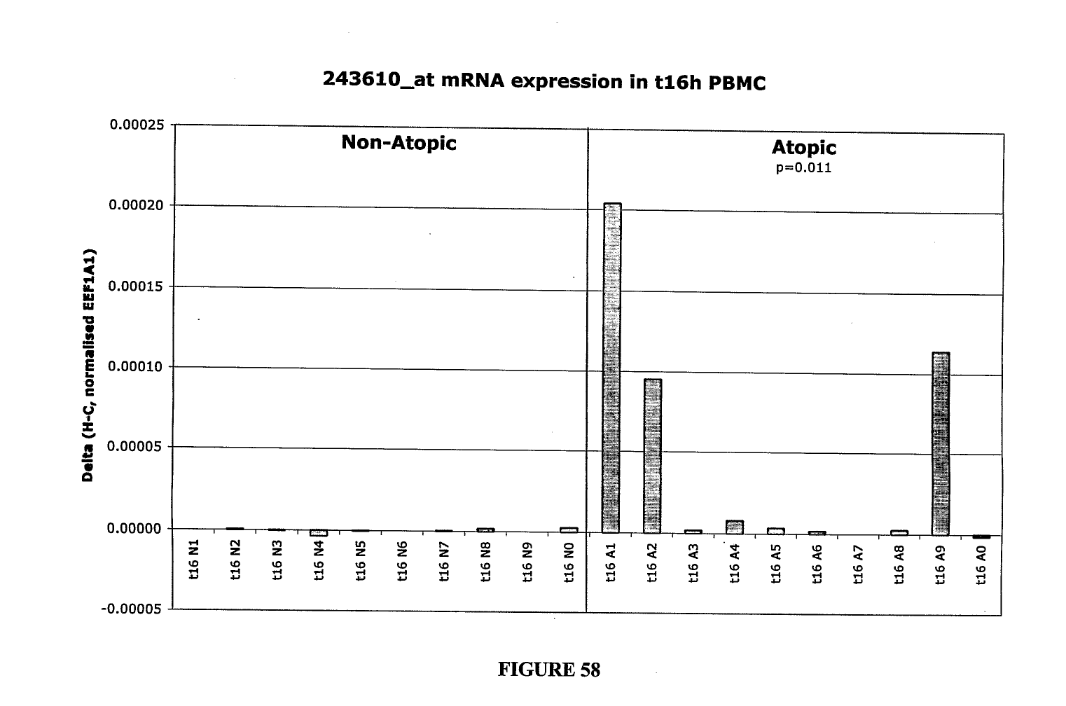

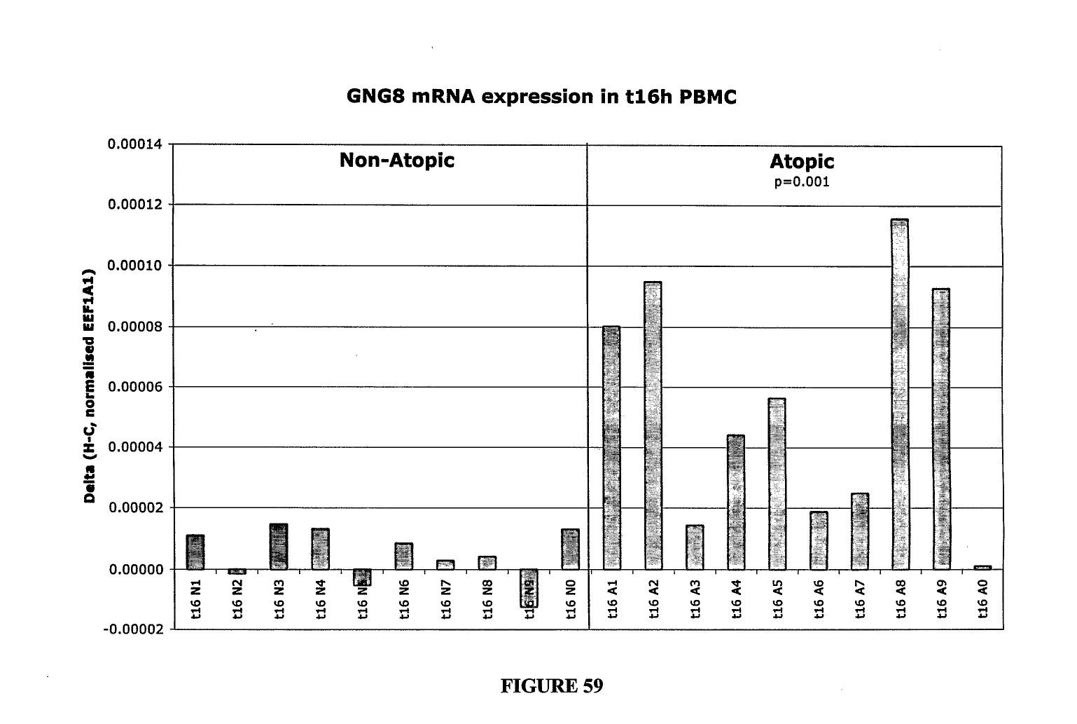

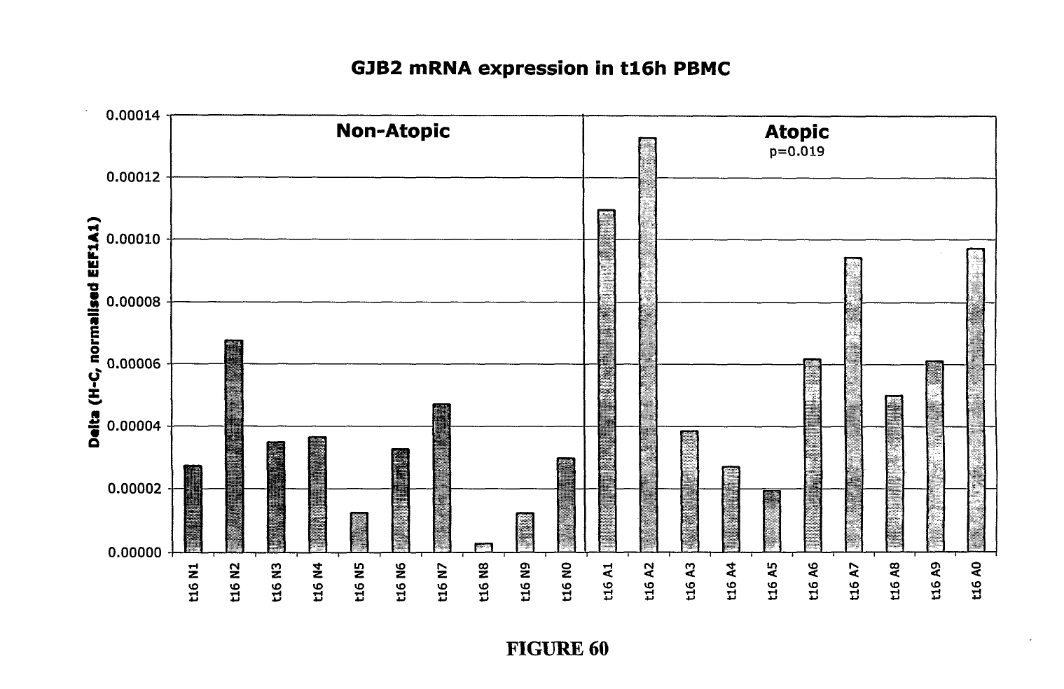

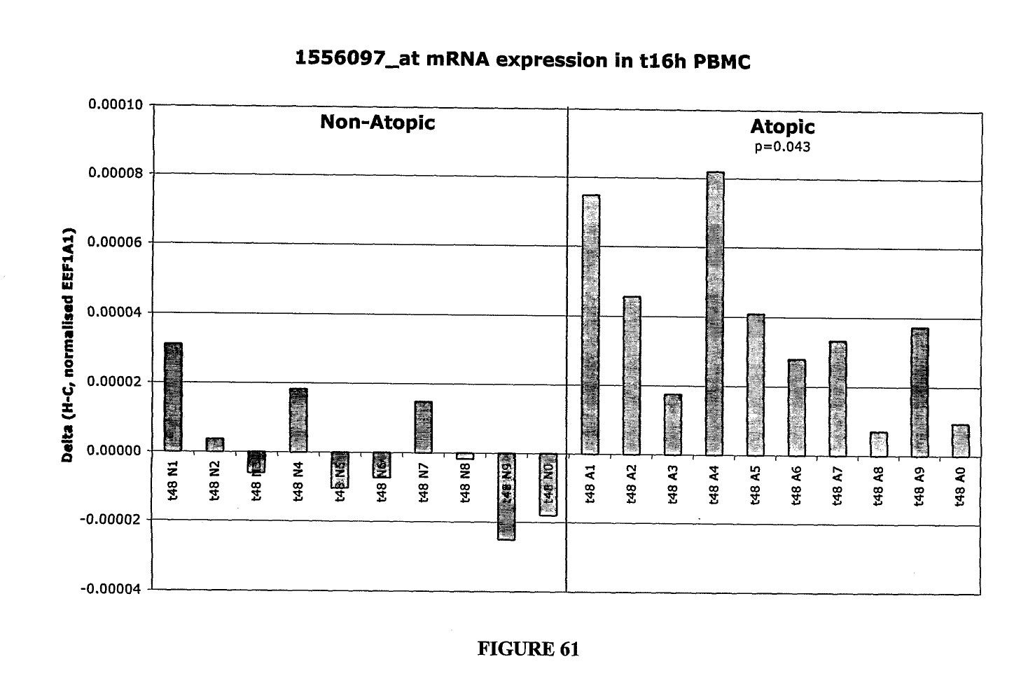

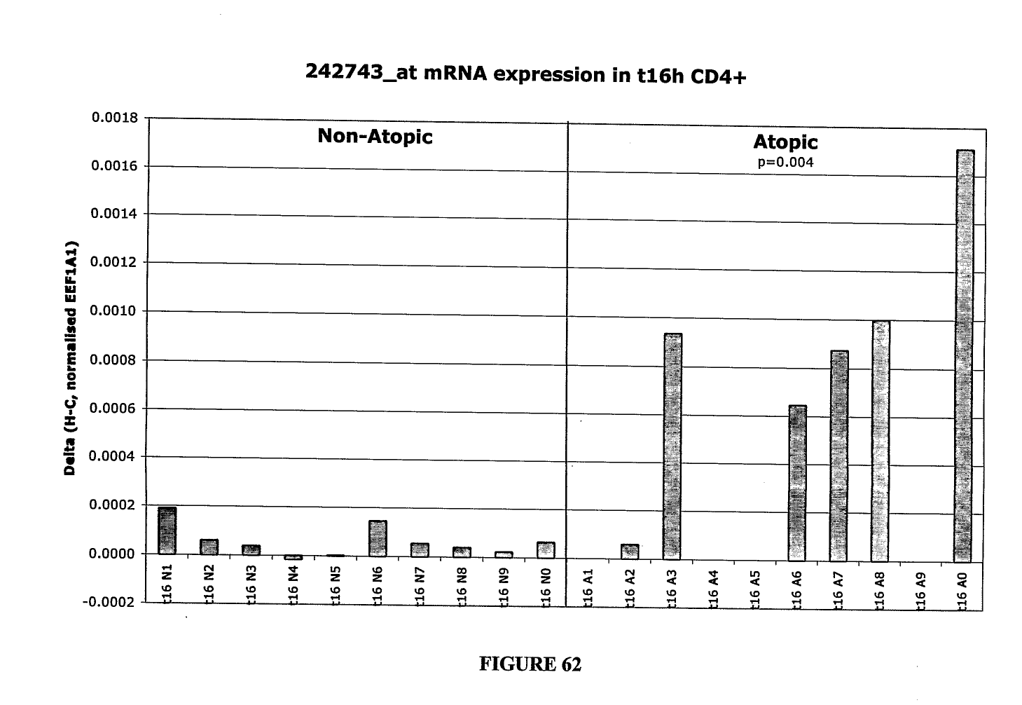

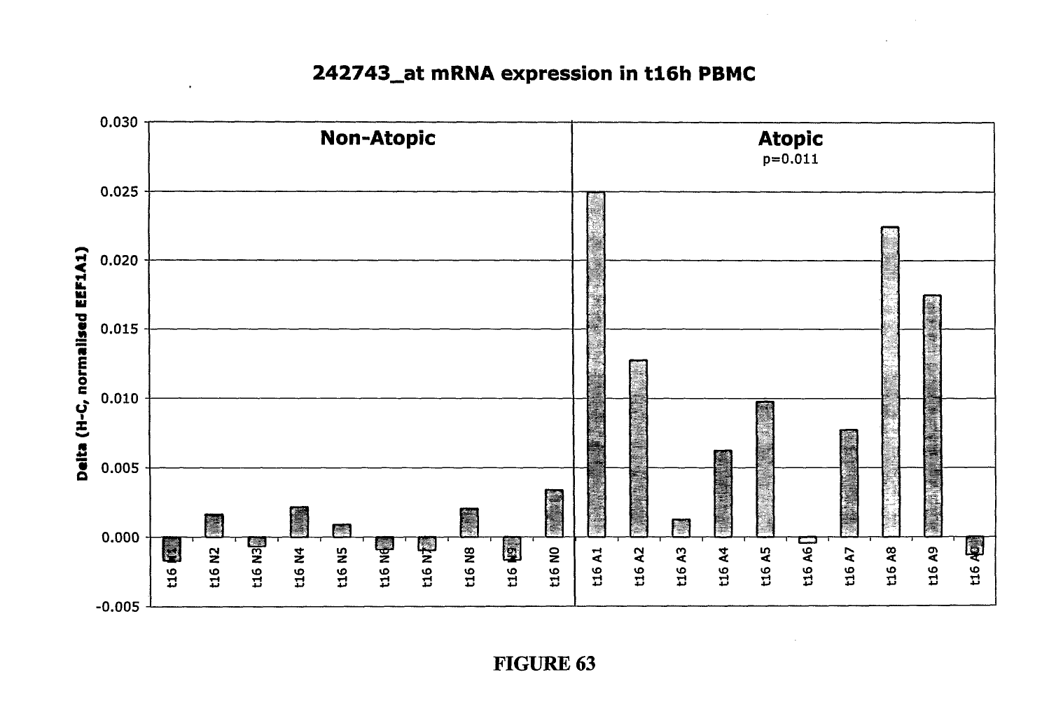

For the validation experiment, RNA from the individual samples employed to generate the pools used for microarray analysis of the PMBC or CD4 kinetic experiments at the 16 and 48 hr time points was converted to cDNA, and then quantitative PCR was performed to detect a series of representative genes. The results are summarised in FIGS. 42 to 63. In some cases a significant change was seen only after 48 hr incubation.

Validation is performed in the same way for the remaining genes, using the following primer sets:

| | |
|---|---|
| IL17RB forward: | |
| 5′TGTGGAGGCACGAAAGGAT3′ | (SEQ ID NO:9) |
| IL17RB reverse: | |
| GATGGGTAAACCACAAGAACCT3′ | (SEQ ID NO:10) |
| KRT1 forward: | |
| 5′TCAATCTCGGTTGGATTCGGA3′ | (SEQ ID NO:11) |
| KRT1 reverse: | |
| 5′CTGCTTGGTAGAGTGCTGTAAGG3′ | (SEQ ID NO:12) |
| LNPEP forward: | |
| 5′TTCACCAATGATCGGCTTCAG3′ | (SEQ ID NO:13) |
| LNPEP reverse: | |
| 5′CTCCATCTCATGCTCACCAAG3′ | (SEQ ID NO:14) |
| OAZ forward: | |
| 5′TCAATTTACACCTGCGATCACTG3′ | (SEQ ID NO:19) |
| OAZ reverse: | |
| 5′GTTGTGGGTCGTCATCACCA3′ | (SEQ ID NO:20) |
| and | |
| RASGRP3 forward: | |
| 5′TCAGCCTCATCGACATATCCA3′ | (SEQ ID NO:25) |
| RASGRP3 reverse: | |
| 5′TCAGCCAATTCAATGGGCTCC3′ | (SEQ ID NO:26) |

It will be apparent to the person skilled in the art that while the invention has been described in some detail for the purposes of clarity and understanding, various modifications and alterations to the embodiments and methods described herein may be made without departing from the scope of the inventive concept disclosed in this specification.

1. A method of diagnosing and/or predicting the development of an allergic disorder in an animal, comprising the step of analysing a biological sample from the animal to determine the level of activation of one or more allergy-associated genes, in which the level of activation is diagnostic of the allergic disorder or predictive of the relative risk for the development of an allergic disorder in the animal.

2. A method of monitoring the progress of therapy of an allergic disorder in an animal undergoing the therapy, comprising the steps of:
a) analysing one or more biological samples from the animal to determine the level of activation of one or more allergy-associated genes, and
b) determining whether the level of activation changes during therapy,
wherein a change in the level of activation during therapy is an indication of the progress of the therapy.

3. A method of determining the potential responsiveness of an individual animal suffering from an allergic disorder to a therapy for the allergic disorder, comprising the step of analysing a biological sample from the animal to determine the level of activation of one or more allergy-associated genes, wherein the level of activation predicts the potential responsiveness of the animal to the therapy.

4. A method of predicting the risk of progression to severe and/or persistent allergy in an animal suffering from an allergic disorder, comprising the step of obtaining a biological sample from the animal and determining the level of mRNA transcripts from one or more allergy-associated genes in the sample, wherein the presence of the mRNA is associated with increased risk of progression to severe and/or persistent allergy.

5. A method of determining the immunological phenotype of an allergic condition in an animal, comprising the steps of obtaining a biological sample from said animal and determining the level of one or more mRNA transcripts from allergy-associated genes in said sample, wherein the presence of the mRNA is associated with or contributes to a specific allergy phenotype.

6. A method of identifying an animal capable of responding to specific immunotherapy, comprising the steps of obtaining a biological sample from the animal, and determining the level of activation of an allergy-associated gene in the sample, wherein the level of activation is predictive of the ability of the animal to respond to immunotherapy.

7. A method of monitoring the response of an allergic animal to immunotherapy, comprising the steps of:
a) analysing one or more biological samples from the animal to determine the level of activation of one or more specific genes encoded by said genes;
b) subjecting the animal to immunotherapy;
c) analysing one or more further biological samples from said animal to determine if the level of activation changes during immunotherapy;
wherein a change in the level of activation during immunotherapy is an indication of the responsiveness of the animal to the immunotherapy.

8. A method of monitoring the effectiveness of a treatment for the reducing the severity of an allergic disorder, comprising the steps of:
a) analysing one or more biological samples from an allergic animal to determine the level of activation of one or more allergy-associated genes;
b) subjecting the animal to the treatment; and
c) analysing one or more further biological samples from the animal to determine if the level of activation changes during treatment;
wherein a change in the level of activation during treatment is an indication of the responsiveness of the animal to the treatment.

9. A method according to claim 1, in which the gene is one or more genes which is selected from the group consisting of cig5, IFIT4, LAMP3, DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 and CISH, or which comprises a sequence selected from the group consisting of sequences identified by probes 243610_at on human chromosome 9q21.13 at locus 138255, 1556097_at on human chromosome 15q25.2 and 242743_at on human chromosome 16p12.1 respectively, or is a combination of two or more of these genes.

10. A method according to claim 1, in which the allergy-associated gene is upregulated in allergen-challenged PBMC from atopic individuals but is upregulated weakly if at all in PBMC from individuals who are not allergic to that allergen.

11. A method according to claim 10, in which the gene is one or more selected from the group consisting of DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 and CISH, and genes which comprise a sequence selected from the group consisting of sequences identified by probes 243610_at on human chromosome 9q21.13 at locus 138255, 1556097_at on human chromosome 15q25.2 and 242743_at on human chromosome 16p12.1 respectively.

12. A method according to claim 10, in which the gene is upregulated in atopic individuals and down-regulated in non-atopic individuals.

13. A method according to claim 12, in which the gene is KRT₁, PECAM₁, PLXDC₁, DACT or MAL.

14. A method according to claim 1, in which the allergy-associated gene is down-regulated in allergen-challenged PBMC from non-atopic individuals, but is not down-regulated in PBMC from atopic individuals.

15. A method according to claim 14, in which the gene is selected from the group consisting of cig5, IFIT4 and LAMP3.

16. A method according to claim 1, in which the step of determining the level of activation of the gene is carried out by detecting the presence of mRNA.

17. A method according to claim 16, in which mRNA is detected using primers specific for a region of one or more genes selected from the group consisting of cig5, IFIT4, LAMP3, DACT1, IL17RB, KRT1, LNPEP, MAL, NCOA3, OAZ, PECAM1, PLXDC1, RASGRP3, SLC39A8, XBP1, NDFIP2, RAB27B, GNG8, GJB2 and CISH, or which comprises a sequence selected from the group consisting of sequences identified by probes 243610_at on human chromosome 9q21.13 at locus 138255, 1556097_at on human chromosome 15q25.2 and 242743_at on human chromosome 16p12.1 respectively.

18. A method according to claim 17, in which the primer is selected from the group consisting of the following sets of primer pairs:

| | |
|---|---|
| cig5 forward: | |
| 5′CAAGACCGGGGAGAATACCTG3′ | (SEQ ID NO:1) |
| cig5 reverse: | |
| 5′GCGAGAATGTCCAAATACTCACC3′ | (SEQ ID NO:2) |
| IFIT4 forward: | |
| 5′GAGTGAGGTCACCAAGAATTC3′ | (SEQ ID NO:3) |
| IFIT4 reverse: | |
| 5′CACTCTATCTTCTAGATCCCTTGAGA3′ | (SEQ ID NO:4) |
| MAL forward: | |
| 5′TCGTGGGTGCTGTGTTTACTCT3′ | (SEQ ID NO:15) |
| MAL reverse: | |
| 5′CAGTTGGAGGTTAGACACAGCAA3′ | (SEQ ID NO:16) |
| NCOA3 forward: | |
| 5′CCTGTCTCAGCCACGAGCTA3′ | (SEQ ID NO:17) |
| NCOA3 reverse: | |
| 5′TCCTGAAAGATCATGTCTGGTAA3′ | (SEQ ID NO:18) |
| PECAM1 forward: | |
| 5′AGTCCAGATAGTCGTATGTGAAATGC3′ | (SEQ ID NO:21) |
| PECAM1 reverse: | |
| GGTCTGTCCTTTTATGACCTCAAAC3′ | (SEQ ID NO:22) |
| SLC39A8 forward: | |
| 5′GCAGTCTTACAGCAATTGAACTTT3′ | (SEQ ID NO:27) |
| SLC39A8 reverse: | |
| 5′CCATATCCCCAAACTTCTGAA3′ | (SEQ ID NO:28) |
| XBP1 forward: | |
| 5′GTAGATTTAGAAGAAGAGAACCAAAAAC3′ | (SEQ ID NO:29) |
| XBP1 reverse: | |
| 5′CCCAAGCGCTGTCTTAACTC3′ | (SEQ ID NO:30) |
| NDFIP2 forward: | |
| 5′AGTGGGGAATGATGGCATTTT3′ | (SEQ ID NO:31) |
| NDFIP2 reverse: | |
| AAATCCGCAGATAGCACCA3′ | (SEQ ID NO:32) |
| RAB27B forward: | |
| 5′CAGAAACTGGATGAGCCAACT3′ | (SEQ ID NO:33) |
| RAB27B reverse: | |
| 5′GACTTCCCTCTGATCTGGTAGG3′ | (SEQ ID NO:34) |
| 243610_at forward: | |
| 5′TGCATTGACAACGTACTCAGAA3′ | (SEQ ID NO:35) |
| 243610_at reverse: | |
| 5′TCATCTTGACAGGGATAAGCAT3′ | (SEQ ID NO:36) |
| GNG8 forward: | |
| 5′GAACATCGACCGCATGAAGGT3′ | (SEQ ID NO:37) |
| GNG8 reverse: | |
| 5′AGAACACAAAAGAGGCGCTTG3′ | (SEQ ID NO:38) |
| GJB2 forward: | |
| 5′GCTTCCTCCCGACGCAGA3′ | (SEQ ID NO:39) |
| GJB2 reverse: | |
| 5′AACGAGGATCATAATGCGAAA3′ | (SEQ ID NO:40) |
| 1556097_at forward: | |
| 5′TCTTATTTCACTTTCTCAACTCATCA3′ | (SEQ ID NO:41) |
| 1556097_at reverse: | |
| 5′GGCATAACCTGAATGTATAATTCAA3′ | (SEQ ID NO:42) |
| 242743_at forward: | |
| 5′GAAAAAGCTGTTGAGTGAAGAAGACT3′ | (SEQ ID NO:43) |
| 242743_at reverse: | |
| 5′TGCAGGATGAGCAATGCTGAGA3′ | (SEQ ID NO:44) |
| and | |
| CISH forward: | |
| 5′GGGAATCTGGCTGGTATTGG3′ | (SEQ ID NO:45) |
| CISH reverse: | |
| 5′TTCTGGCATCTTCTGCAGGTGTT3′. | (SEQ ID NO:46) |

19. A method according to claim 16, in which protein encoded by the mRNA is detected.


SEQUENCE LISTING (48 sequences)

SEQ ID NO: 1 - DNA, 21 bp, Homo sapiens
caagaccggggagaatacctg

SEQ ID NO: 2 - DNA, 23 bp, Homo sapiens
gcgagaatgtccaaatactcacc

SEQ ID NO: 3 - DNA, 21 bp, Homo sapiens
gagtgaggtcaccaagaattc

SEQ ID NO: 4 - DNA, 26 bp, Homo sapiens
cactctatcttctagatcccttgaga

SEQ ID NO: 5 - DNA, 19 bp, Homo sapiens
gcgtccctggccgtaattt

SEQ ID NO: 6 - DNA, 20 bp, Homo sapiens
tggttgcttagctggttgct

SEQ ID NO: 7 - DNA, 21 bp, Homo sapiens
aactcggtgttcagtgagtgt

SEQ ID NO: 8 - DNA, 19 bp, Homo sapiens
ggagagggaacggcaaact

SEQ ID NO: 9 - DNA, 19 bp, Homo sapiens
tgtggaggcacgaaaggat

SEQ ID NO: 10 - DNA, 22 bp, Homo sapiens
gatgggtaaaccacaagaacct

SEQ ID NO: 11 - DNA, 21 bp, Homo sapiens
tcaatctcggttggattcgga

SEQ ID NO: 12 - DNA, 23 bp, Homo sapiens
ctgcttggtagagtgctgtaagg

SEQ ID NO: 13 - DNA, 21 bp, Homo sapiens
ttcaccaatgatcggcttcag

SEQ ID NO: 14 - DNA, 21 bp, Homo sapiens
ctccatctcatgctcaccaag

SEQ ID NO: 15 - DNA, 22 bp, Homo sapiens
tcgtgggtgctgtgtttactct

SEQ ID NO: 16 - DNA, 23 bp, Homo sapiens
cagttggaggttagacacagcaa

SEQ ID NO: 17 - DNA, 20 bp, Homo sapiens
cctgtctcagccacgagcta

SEQ ID NO: 18 - DNA, 23 bp, Homo sapiens
tcctgaaagatcatgtctggtaa

SEQ ID NO: 19 - DNA, 23 bp, Homo sapiens
tcaatttacacctgcgatcactg

SEQ ID NO: 20 - DNA, 20 bp, Homo sapiens
gttgtgggtcgtcatcacca

SEQ ID NO: 21 - DNA, 26 bp, Homo sapiens
agtccagatagtcgtatgtgaaatgc

SEQ ID NO: 22 - DNA, 25 bp, Homo sapiens
ggtctgtccttttatgacctcaaac

SEQ ID NO: 23 - DNA, 19 bp, Homo sapiens
cctgggcatgtgtcagagc

SEQ ID NO: 24 - DNA, 22 bp, Homo sapiens
ggtgttggagagtattgtgtgg

SEQ ID NO: 25 - DNA, 21 bp, Homo sapiens
tcagcctcatcgacatatcca

SEQ ID NO: 26 - DNA, 21 bp, Homo sapiens
tcagccaattcaatgggctcc

SEQ ID NO: 27 - DNA, 24 bp, Homo sapiens
gcagtcttacagcaattgaacttt

SEQ ID NO: 28 - DNA, 21 bp, Homo sapiens
ccatatccccaaacttctgaa

SEQ ID NO: 29 - DNA, 28 bp, Homo sapiens
gtagatttagaagaagagaaccaaaaac

SEQ ID NO: 30 - DNA, 20 bp, Homo sapiens
cccaagcgctgtcttaactc

SEQ ID NO: 31 - DNA, 21 bp, Homo sapiens
agtggggaatgatggcatttt

SEQ ID NO: 32 - DNA, 19 bp, Homo sapiens
aaatccgcagatagcacca

SEQ ID NO: 33 - DNA, 21 bp, Homo sapiens
cagaaactggatgagccaact

SEQ ID NO: 34 - DNA, 22 bp, Homo sapiens
gacttccctctgatctggtagg

SEQ ID NO: 35 - DNA, 22 bp, Homo sapiens
tgcattgacaacgtactcagaa

SEQ ID NO: 36 - DNA, 22 bp, Homo sapiens
tcatcttgacagggataagcat

SEQ ID NO: 37 - DNA, 21 bp, Homo sapiens
gaacatcgaccgcatgaaggt

SEQ ID NO: 38 - DNA, 21 bp, Homo sapiens
agaacacaaaagaggcgcttg

SEQ ID NO: 39 - DNA, 18 bp, Homo sapiens
gcttcctcccgacgcaga

SEQ ID NO: 40 - DNA, 21 bp, Homo sapiens
aacgaggatcataatgcgaaa

SEQ ID NO: 41 - DNA, 26 bp, Homo sapiens
tcttatttcactttctcaactcatca

SEQ ID NO: 42 - DNA, 25 bp, Homo sapiens
ggcataacctgaatgtataattcaa

SEQ ID NO: 43 - DNA, 26 bp, Homo sapiens
gaaaaagctgttgagtgaagaagact

SEQ ID NO: 44 - DNA, 22 bp, Homo sapiens
tgcaggatgagcaatgctgaga

SEQ ID NO: 45 - DNA, 20 bp, Homo sapiens
gggaatctggctggtattgg

SEQ ID NO: 46 - DNA, 23 bp, Homo sapiens
ttctggcatcttctgcaggtgtt

SEQ ID NO: 47 - DNA, 24 bp, Homo sapiens
aacagcctcacagagcagaagact

SEQ ID NO: 48 - DNA, 22 bp, Homo sapiens
cagcgagtgtccttctcatggt